(19) United States
(12) Patent Application Publication
(10) Pub. No.: US 2023/0382948 A1
(43) Pub. Date: Nov. 30, 2023

(54) DELIVERY OF ENDOTHELIAL CELL-LADEN MICROGEL ELICITS ANGIOGENESIS IN SELF-ASSEMBLING ULTRASHORT PEPTIDE HYDROGELS

(71) Applicant: KING ABDULLAH UNIVERSITY OF SCIENCE AND TECHNOLOGY, Thuwal (SA)

(72) Inventors: Charlotte A. E. HAUSER, Thuwal (SA); Gustavo Andres RAMIREZ-CALDERON, Thuwal (SA)

(21) Appl. No.: 18/032,351

(22) PCT Filed: Oct. 20, 2021
(86) PCT No.: PCT/IB2021/059652

§ 371 Date: Apr. 18, 2023

Related U.S. Application Data
(60) Provisional application No. 63/094,377, filed on Oct. 21, 2020.

(30) Foreign Application Priority Data
Oct. 20, 2021 (WO) 63094377

(51) Int. Cl. C07K 5/103; C07K 5/11; C07K 5/113; C12N 5/00

(52) U.S. Cl. CPC C07K 5/1013; C07K 5/1019; C07K 5/1021; C12N 5/0068; C12N 2531/00; C12N 2533/50; C12N 2535/00

(57) ABSTRACT

The present disclosure relates to a cell-laden microgel comprising self-assembly ultrashort peptide (SUP) and a method of frabricating such cell-laden microgels. The present disclosure also relates to a cell microcarrier comprising cell-laden microgels, which is suitable for medical applications such as cell therapy. The present disclosure further relates to a system comprising a combination of SUP microgel and SUP bulk hydrogel for vascularized tissue culture and a method of creating such a vascularized 3D tissue constructs with improved cell viability and proliferation.

### REFERENCE TO A “SEQUENCE LISTING”

The present application includes a Sequence Listing which has been submitted electronically in an ASCII text format. This Sequence Listing is named 114147-23871US0 l_sequence listing.TXT was created on Oct. 18, 2021, is 7,808 bytes in size and is hereby incorporated by reference in its entirety.

### BACKGROUND

### Field of the Invention

The present disclosure relates to a cell-laden microgel comprising self-assembly ultrashort peptide (SUP) and a method of frabricating such cell-laden microgels. The present disclosure also relates to a cell microcarrier comprising cell-laden microgels, which is suitable for medical applications such as cell therapy. The present disclosure further relates to a system comprising a combination of SUP microgel and SUP bulk hydrogel for vascularized tissue culture and a method of creating such a vascularized 3D tissue constructs with improved cell viability and proliferation.

### Background of the Invention

Cardiovascular diseases are causing the highest numbers of fatalities with more than 30% of global deaths. 1 The disease development can lead to several organic and tissue failures due to insufficient blood supply. Regenerative medicine and tissue engineering aim to develop therapies to generate new blood vessels that restore the flow in ischemic tissue and enhance tissue regeneration. 2,3 However, the manufacturing of vascularized tissue remains a challenge, even with new technologies such as 3D bioprinting. 4 The traditional top-down fabrication techniques do not recreate the native tissue microarchitecture.

Recently, sacrificial ink has become increasingly popular to create a vascular channel using bioprinting, though the approach itself does not recreate the native tissue microarchitecture. 5,6 Therefore, modular tissue engineering can be applied to create more biomimetic engineered tissue.⁷ Modular tissue engineering is a bottom-up approach to produce vascularized organotypic structures with micro-organization driven by pre-formed repetitive units. 8-10 In this approach, the use of extracellular matrix (ECM)-like materials is critical for cell growth and differentiation in a tissue-like environment. The materials should be biocompatible, support vascularization and have high translational potential.¹¹⁻¹³ A minimalist use of materials that satisfies these requirements is best as it decreases the complexity and the material limitations. Thus, the use of a highly defined material is ideal. Current fabrication techniques and biomaterials lack translational potential in medicine. Naturally-derived biomaterials harbor the risk of immunogenicity and pathogen transmission, while synthetic materials need functionalization or blending to improve their biocompatibility. Therefore, there is a need for improved materials that satisfy the above requirements.

### SUMMARY

According to a first broad aspect the present disclosure provides a cell-laden microgel comprising: at least one self-assembly ultrashort peptide (SUP) scaffold; and at least one mammalian cells, wherein the microgel has a spherical shape, and wherein the diameter of the microgel is 100-900 μm.

According to a second broad aspect the present disclosure provides a method of fabricating cell-laden microgel comprising: feeding a microfluidic flow-focusing chip with at least one self-assembly ultrashort peptide (SUP) solution through a first inlet; feeding a microfluidic flow-focusing chip with oil through a second inlet; fabricating cell-free microgel using the microfluidic flow-focusing chip; and loading the cell-free microgel with at least one mammalian cells, wherein the oil comprising at least one selected from the group consisting of salt and surfactant, wherein the microgel has a spherical shape, and wherein the diameter of the microgel is 100-900 μm.

According to a third broad aspect the present disclosure provides a cell culture system comprising: at least one cell-laden microgels; and at least one cell-loaded bulk hydrogels, wherein the cell-laden microgels comprises a first self-assembly ultrashort peptide (SUP) scaffold and a first mammalian cell, wherein the microgel has a spherical shape, wherein the diameter of the microgel is 100-900 μm, and wherein the cell-loaded bulk hydrogels comprises a second self-assembly ultrashort peptide (SUP) scaffold and a second mammalian cell.

According to a fourth broad aspect the present disclosure provides a method of creating SUP-based cell culture system comprising: feeding a microfluidic flow-focusing chip with a first self-assembly ultrashort peptide (SUP) solution through a first inlet; feeding a microfluidic flow-focusing chip with oil through a second inlet; fabricating cell-free microgel using the microfluidic flow-focusing chip; loading the cell-free microgel with a first mammalian cells to create cell-laden microgels; and dispersing the cell-laden microgels in a bulk hydrogel comprising a second self-assembly ultrashort peptide (SUP) scaffold and a second mammalian cell, wherein the oil comprising at least one selected from the group consisting of salt and surfactant, wherein the microgel has a spherical shape, and wherein the diameter of the microgel is 100-900 μm.

Other aspects and features of the present disclosure will become apparent to those skilled in the art upon review of the following description of specific embodiments of the invention in conjunction with the accompanying figures.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 is a graph showing liquid chromatograms by the absorbance at 220 nm of IVFK (SEQ ID NO. 1) according to an embodiment of the present disclosure.

FIG. 2 is a graph showing liquid chromatograms by the absorbance at 220 nm of IVZK (SEQ ID NO. 2) according to an embodiment of the present disclosure.

FIG. 3 is a graph showing mass spectrum of IVFK (SEQ ID NO. 1) according to an embodiment of the present disclosure.

FIG. 4 is a graph showing mass spectrum of IVZK (SEQ ID NO. 2) according to an embodiment of the present disclosure.

FIG. 5 is a graph showing ¹H-NMR analysis of IVFK (SEQ ID NO. 1) according to an embodiment of the present disclosure.

FIG. 6 is a graph showing ¹H-NMR analysis of IVZK (SEQ ID NO. 2) according to an embodiment of the present disclosure.

FIG. 7 is a graph showing scheme of self-assembling ultrashort peptides IVFK (SEQ ID NO. 1) and IVZK (SEQ ID NO. 2) according to an embodiment of the present disclosure.

FIG. 8 is a graph showing the angiogenesis model using endothelial cell (EC) laden IVFK (SEQ ID NO. 1) and IVZK (SEQ ID NO. 2) microgels according to an embodiment of the present disclosure.

FIG. 9 is a photo showing the characterization of gelation time of IVFK (SEQ ID NO. 1) and IVZK (SEQ ID NO. 2) peptides according to an embodiment of the present disclosure.

FIG. 10 is a photo showing the topography of IVFK (SEQ ID NO. 1) and IVZK (SEQ ID NO. 2) hydrogel according to an embodiment of the present disclosure.

FIG. 11 is a graph showing the FTIR absorption spectra of IVFK (SEQ ID NO. 1) and IVZK (SEQ ID NO. 2) according to an embodiment of the present disclosure.

FIG. 12 is TEM micrographs of IVFK (SEQ ID NO. 1) and IVZK (SEQ ID NO. 2) showing the entangled nanofibrous network according to an embodiment of the present disclosure.

FIG. 13 is a graph showing the fiber thickness of IVFK (SEQ ID NO. 1) network according to an embodiment of the present disclosure.

FIG. 14 is a graph showing the fiber thickness of IVZK (SEQ ID NO. 2) network according to an embodiment of the present disclosure.

FIG. 15 is a graph showing the storage moduli of IVFK (SEQ ID NO. 1) and IVZK (SEQ ID NO. 2) hydrogels at different concentrations according to an embodiment of the present disclosure.

FIG. 16 is a graph showing the G′ and G″ of IVFK (SEQ ID NO. 1) as a function of time (time sweep) at 1 rads and 0.1% strain according to an embodiment of the present disclosure.

FIG. 17 is a graph showing the G′ and G″ of IVZK (SEQ ID NO. 2) as a function of time (time sweep) at 1 rads and 0.1% strain according to an embodiment of the present disclosure.

FIG. 18 is a graph showing the G′ and G″ of IVFK (SEQ ID NO. 1) as a function of strain (amplitude sweep) at 1 rads and 0.1% strain according to an embodiment of the present disclosure.

FIG. 19 is a graph showing the G′ and G″ of IVZK (SEQ ID NO. 2) as a function of strain (amplitude sweep) at 1 rads and 0.1% strain according to an embodiment of the present disclosure.

FIG. 20 is a graph showing the G′ and G″ of IVFK (SEQ ID NO. 1) as a function of angular frequency (frequency sweep) at 1 rads and 0.1% strain according to an embodiment of the present disclosure.

FIG. 21 is a graph showing the G′ and G″ of IVZK (SEQ ID NO. 2) as a function of angular frequency (frequency sweep) at 1 rad/s and 0.1% strain according to an embodiment of the present disclosure.

FIG. 22 is a photo showing the microfluidic setup according to an embodiment of the present disclosure.

FIG. 23 is a photo showing the microscopic characterization of SUP microgels according to an embodiment of the present disclosure.

FIG. 24 is a photo showing the merged SUP microgels according to an embodiment of the present disclosure.

FIG. 25 is a photo showing the fused SUP droplets according to an embodiment of the present disclosure.

FIG. 26 is a photo showing the 100 nm green FluoSpheres incorporated in fused SUP solution according to an embodiment of the present disclosure.

FIG. 27 is a photo showing the SEM images of IVFK (SEQ ID NO. 1) and IVZK (SEQ ID NO. 2) microgels according to an embodiment of the present disclosure.

FIG. 28 is a photo showing the effect of SUP concentration on microgel integrity according to an embodiment of the present disclosure.

FIG. 29 is a photo showing the microgel diameter in oil or PBS for IVFK (SEQ ID NO. 1) and IVZK (SEQ ID NO. 2) according to an embodiment of the present disclosure.

FIG. 30 is a photo showing the microgel roundness in oil or PBS for IVFK (SEQ ID NO. 1) and IVZK (SEQ ID NO. 2) according to an embodiment of the present disclosure.

FIG. 31 is a graph showing the experimental design scheme to test SUP microgel stability challenged against culturing procedures according to an embodiment of the present disclosure.

FIG. 32 is a graph showing the IVFK (SEQ ID NO. 1) microgel diameter challenged with various culture procedure according to an embodiment of the present disclosure.

FIG. 33 is a graph showing the IVFK (SEQ ID NO. 1) microgel roundness challenged with various culture procedure according to an embodiment of the present disclosure.

FIG. 34 is a graph showing the 1VFK (SEQ ID NO. 1) microgel diameter at 22° C. along time according to an embodiment of the present disclosure.

FIG. 35 is a graph showing the IVFK (SEQ ID NO. 1) microgel roundness at 22° C. along time according to an embodiment of the present disclosure.

FIG. 36 is a graph showing the IVFK (SEQ ID NO. 1) microgel diameter at 37° C. along time according to an embodiment of the present disclosure.

FIG. 37 is a graph showing the IVFK (SEQ ID NO. 1) microgel roundness at 37° C. along time according to an embodiment of the present disclosure.

FIG. 38 is a graph showing the IVFK (SEQ ID NO. 1) microgel diameter at 37° C. on shaking along time according to an embodiment of the present disclosure.

FIG. 39 is a graph showing the IVFK (SEQ ID NO. 1) microgel roundness at 37° C. on shaking along time according to an embodiment of the present disclosure.

FIG. 40 is a graph showing the IVZK (SEQ ID NO. 2) microgel diameter challenged with various culture procedure according to an embodiment of the present disclosure.

FIG. 41 is a graph showing the IVZK (SEQ ID NO. 2) microgel roundness challenged with various culture procedure according to an embodiment of the present disclosure.

FIG. 42 is a graph showing the IVZK (SEQ ID NO. 2) microgel diameter at 22° C. along time according to an embodiment of the present disclosure.

FIG. 43 is a graph showing the IVZK (SEQ ID NO. 2) microgel roundness at 22° C. along time according to an embodiment of the present disclosure.

FIG. 44 is a graph showing the IVZK (SEQ ID NO. 2) microgel diameter at 37° C. along time according to an embodiment of the present disclosure.

FIG. 45 is a graph showing the IVZK (SEQ ID NO. 2) microgel roundness at 37° C. along time according to an embodiment of the present disclosure.

FIG. 46 is a graph showing the IVZK (SEQ ID NO. 2) microgel diameter at 37° C. on shaking along time according to an embodiment of the present disclosure.

FIG. 47 is a graph showing the IVZK (SEQ ID NO. 2) microgel roundness at 37° C. on shaking along time according to an embodiment of the present disclosure.

FIG. 48 is a photo showing HeLa attachment on the SUP microgels according to an embodiment of the present disclosure.

FIG. 49 is a photo showing HDFn cell laden IVFK (SEQ ID NO. 1) microgel after 24 h of culture according to an embodiment of the present disclosure.

FIG. 50 is a photo showing HUVEC cell laden IVZK (SEQ ID NO. 2) microgel after 24 h of culture according to an embodiment of the present disclosure.

FIG. 51 is a photo showing HUVEC cell laden IVZK (SEQ ID NO. 2) microgel after 8 days of culture according to an embodiment of the present disclosure.

FIG. 52 is a photo showing HDFn cell laden IVFK (SEQ ID NO. 1) microgel after 8 days of culture according to an embodiment of the present disclosure.

FIG. 53 is a photo showing HDFn cell bridges clump SUP microgels according to an embodiment of the present disclosure.

FIG. 54 is a photo showing vascular network formation in 3D SUP matrices using a SUP microgel-based angiogenic in vitro assay according to an embodiment of the present disclosure.

FIG. 55 is a photo showing vascular networks co-localize with fibroblast in 3D SUP matrices according to an embodiment of the present disclosure.

FIG. 56 is a photo showing stiff SUP hydrogel hinders endothelial sprouting according to an embodiment of the present disclosure.

FIG. 57 is a graph showing the time required for IIFK (SEQ ID NO. 33), IIZK (SEQ ID NO. 34) and IZZK (SEQ ID NO. 35) to form a gel at different concentration according to an embodiment of the present disclosure.

FIG. 58 is a graph showing the cell viability of HDFn within IIFK (SEQ ID NO. 33) and IIZK (SEQ ID NO. 34) peptide hydrogels according to an embodiment of the present disclosure.

FIG. 59 is a graph showing the cell mophology of HDFn within IIFK (SEQ ID NO. 33) and IIZK (SEQ ID NO. 34) peptide hydrogels versus 2D culture according to an embodiment of the present disclosure.

FIG. 60 is a graph showing the HDFn proliferation through quantitation of ATP production according to an embodiment of the present disclosure.

### DETAILED DESCRIPTION OF THE INVENTION

### Definitions

Where the definition of terms departs from the commonly used meaning of the term, applicant intends to utilize the definitions provided below, unless specifically indicated.

Unless defined otherwise, all technical and scientific terms used herein have the same meaning as is commonly understood to which the claimed subject matter belongs. In the event that there is a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the Internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

For purposes of the present disclosure, the term “comprising”, the term “having”, the term “including,” and variations of these words are intended to be open-ended and mean that there may be additional elements other than the listed elements.

For purposes of the present disclosure, directional terms such as “top,” “bottom,” “upper,” “lower,” “above,” “below,” “left,” “right,” “horizontal,” “vertical,” “up,” “down,” etc., are used merely for convenience m describing the various embodiments of the present disclosure. The embodiments of the present disclosure may be oriented in various ways. For example, the diagrams, apparatuses, etc., shown in the drawing figures may be flipped over, rotated by 90° in any direction, reversed, etc.

For purposes of the present disclosure, a value or property is “based” on a particular value, property, the satisfaction of a condition, or other factor, if that value is derived by performing a mathematical calculation or logical decision using that value, property or other factor.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

For purposes of the present disclosure, the term “amphiphilic” or “amphiphilicity” refers to being a compound consisting of molecules having a water-soluble group at one end and a water-insoluble group at the other end.

The term “aliphatic” means, unless otherwise stated, a straight or branched hydrocarbon chain, which may be saturated or mono- or poly-unsaturated and include heteroatoms. An unsaturated aliphatic group contains one or more double and/or triple bonds (alkenyl or alkynyl moieties). The branches of the hydrocarbon chain may include linear chains as well as non-aromatic cyclic elements. The hydrocarbon chain, which may, unless otherwise stated, be of any length, and contain any number of branches. Typically, the hydrocarbon (main) chain includes 1 to 5, to 10, to 15 or to 20 carbon atoms. Examples of alkenyl radicals are straight-chain or branched hydrocarbon radicals which contain one or more double bonds. Alkenyl radicals generally contain about two to about twenty carbon atoms and one or more, for instance two, double bonds, such as about two to about ten carbon atoms, and one double bond. Alkynyl radicals normally contain about two to about twenty carbon atoms and one or more, for example two, triple bonds, preferably such as two to ten carbon atoms, and one triple bond. Examples of alkynyl radicals are straight-chain or branched hydrocarbon radicals which contain one or more triple bonds. Examples of alkyl groups are methyl, ethyl, propyl, butyl, pentyl, hexyl, heptyl, octyl, nonyl, decyl, the n isomers of these radicals, isopropyl, isobutyl, isopentyl, sec-butyl, tert-butyl, neopentyl, 3,3 dimethylbutyl. Both the main chain as well as the branches may furthermore contain heteroatoms as for instance N, O, S, Se or Si or carbon atoms may be replaced by these heteroatoms.

For purposes of the present disclosure, the term “bioinks” as used herein means materials used to produce engineered/artificial live tissue, cellular grafts and organ substitutes (organoids) using 3D printing. In the present disclosure, these bioinks are mostly composed of hydrogel or organogel with cellular components embedded.

For purposes of the present disclosure, the term “gel” refers to both “hydrogel” and “organogel”. These terms refer to a is a network of polymer chains, entrapping water or other aqueous solutions, such as physiological buffers, of over 99% by weight. In an embodiment of the present disclosure, the polymer chains may be a peptide with repetitive sequences. If the self-assembly of the ultrashort peptides occurs in aqueous solution, hydrogels are formed. If organic solvents are used, organogels are formed. The term “microgel” refer hydrogels with spherical shape at the micro-scale. The term “bulk hydrogel” refers to hydrogels with greater than micro-scale diameter.

For purposes of the present disclosure, the term “PBS” refers to a buffer solution commonly used in biological research, which is an abbreviation of phosphate-buffered saline. It is a water-based salt solution, helping to maintain a constant pH, as well as osmolarity and ion concentrations to match those of most cells. In some embodiments, PBS may include a water-based salt solution containing disodium hydrogen phosphate, sodium chloride and, in some formulations, potassium chloride and potassium dihydrogen phosphate.

For purposes of the present disclosure, the term “printability” refers to the suitability of peptide for 3D printing. In particular, it refers to the suitable speed of self-assembly at certain concentration, and viscosity. The speed of forming gel and viscosity need to be high enough so that a structure with certain height can be printed without collapsing. On the other hand, the speed and viscosity need to be low enough so that the peptide will not clog the nozzle of bioprinters.

For purposes of the present disclosure, the term “scaffolds” as used herein means the supramolecular network structures made from self-assembling ultra-short peptide or other polymer materials in the bioinks that provide support for the cellular components.

For purposes of the present disclosure, the term “structure fidelity” refers to the ability of 3D constructs to maintain its shape and internal structure over time.

For purposes of the present disclosure, the term “ultra-short peptide” refers to a sequence containing 3-7 amino acids. The peptides according an aspect of the present disclosure are also particularly useful for formulating aqueous or other solvent compositions, herein also sometimes referred to as “inks” or “bioinks” when mixed with cellular components, which may be used as inks for printing structures and as bioinks for printing cellular or tissue structures, in particular 3D structures. Such printed structures make use of the gelation properties of the peptides according to features of the present disclosure.

For purposes of the present disclosure, the terms “biocompatible” (which also can be referred to as “tissue compatible”) and “biocompatibility”, as used herein, refer to the property of a hydrogel that produces little if any adverse biological response when used in vivo.

For purposes of the present disclosure, the terms “v/v”, “v/v %” and “% v/v” are used interchangeably. These terms refer to Volume concentration of a solution.

For purposes of the present disclosure, the terms “w/v”, “w/v %” and “% w/v” are used interchangeably. These terms refer to Mass concentration of a solution, which is expressed as weight per volume.

### DESCRIPTION

In one embodiment, the self-assembling ultrashort peptides (SUPs) are tri- to hexapeptides with a characteristic amphiphilic motif of a hydrophobic tail capped by a polar head.¹⁴ This sequence motif drives the peptide self-assembling to supramolecular structures resembling fibers, then bundles and finally, networks that mimic the ECM.

In one embodiment, the ECM-like feature makes SUP a suitable biomaterial for cell culture applications in a chemically defined natural-like environment.^{15,16} Consequently, SUPs have been used in tissue engineering and regenerative medicine as cell-laden hydrogels that can be casted in almost any shape retaining up to hundreds of times their dry weight in water.^{12, 15, 17-21} Notwithstanding, these applications had been using hydrogels of relatively large size (millimeter to centimeter scale) that are not useful for a bottom-up tissue assembly because native tissue has repeating functional units on submillimeter scale.⁹ The challenge in developing a microgel using a SUP lies in cutting down on hydrogel size without losing its physicochemical and biocompatible features through a cost-effective methodical approach. Most of the currently available microgels have known drawbacks coming from their material and fabrication method.^{10, 22, 23} For instance, naturally derived materials such as collagen and alginate have batch-to-batch variation, potential immunogenicity causing by either itself or side products such as lipopolysaccharides and lack appropriate mechanical strength, while synthetic polymers need functionalization or blending with natural products to improve their cell adhesion and biocompatibility. Selection of the most suited microgel fabrication method relies mainly on material cross-linking properties, with thermal, photo, ionic and chemical crosslinking methods being among the most commonly used for microgel production. Photo-and thermal sensitivity exclude the use of common sterilization techniques such as autoclaving and UV-irradiation, while ionic and chemical crosslinking could affect cell signaling, which uses Ca²⁺ as one of secondary messengers and viability, respectively.

In one embodiment, SUP is a synthetic peptide composed of naturally derived amino acids. After self-assembly, the SUP hydrogel combines the qualities of both material types, showing optimal performance in biocompatibility and mechanical strength.^{15,16}

In one embodiment, SUP is a class of amphiphilic linear peptide, containing a hydrophobic tail and hydrophilic headgroup. At physiological conditions, SUP can self-assemble to form a three-dimensional nanofiber hydrogel scaffold that closely resemble fibers within the extracellular matrix.¹⁸ The assembly is driven by non-covalent interactions such as hydrophobic interaction, hydrogen bonding, and electrostatic interaction.¹⁴ Furthermore, as the production of peptide with specific sequence can be achieved based on well-established peptide chemistry, SUP can be easily produced, modified and upscaled, using the sequences provided in the present disclosure. Moreover, it has a very low immunogenicity risk due to its ultra-short size that makes it almost irrecognizable by the immune system. 17,24,25

Microgel production using self-assembling peptides is still not widely done because of a variety of difficulties. For example, optimization of the gelation time during fabrication is problematic because gelation occurs too fast and thus clogs the microfluidics chip or too slow, thus merging microgels, losing shape fidelity, and showing a wide size distribution. Microgel fabrication based solely on peptides has been done using peptide sequences of 16 and 11 amino acids, such as RADA16 and Q11. In these cases, ionic strength-sensitive assembly in emulsion processing or microfluidics for fabrication was used.^{26, 27} The latter was showing better control and narrower distribution.

In one embodiment, the fabrication of microgels can be from self assembling ultrashort tetrameric peptides. The microgels comprising self assembling ultrashort peptides can be uses as endothelial microcarriers to trigger vascularization in SUP hydrogels loaded with fibroblasts.

In one embodiment, microgels were fabricated using at least one of two different SUPS gelling in a water-in-oil emulsion in a microfluidic droplet generator chip.

In one embodiment, SUP microgels are round with a diameter of 300-350 μm and ECM-like topography.

In one embodiment, SUP microgels have a long shelf life once stored in water and are stable under culture conditions without changing either the size or the shape.

In one embodiment, the SUP microgels were used as microcarriers to grow HUVECS and HDFn cells on the microgel surface, showing cell attachment, stretching, and proliferation.

In one embodiment, the endothelial cell-laden SUP microgel was suitable for generating endothelial networks within a SUP hydrogel carrying HDFn cells, similarly to current angiogenesis in vitro assays.^{28, 29}

In one embodiment, this in vitro 3D tissue construct model made of SUP microgel can be used to study angiogenesis and may be used for cell therapies to develop vascularization in ischemic tissue.

### SUP Design and Structural Features

In one embodiment, peptides IVFK (SEQ ID NO. 1) and IVZK (SEQ ID NO. 2) were synthesized following self-assembling peptide design rules and based on the shortest SUPs IVF and IVK.^{14,15,32,33} The self-assembling feature of these SUP resuires the peptides to be amphiphilic, consisting of a tail of aliphatic nonpolar amino acids at the N terminus with decreasing hydrophobicity and a hydrophilic head group of acidic, neutral, or basic nonaromatic polar amino acids (C terminus). The length of the hydrophobic tail and the polarity of the head group were integral elements that supported facile hydrogel formation.

In one embodiment, the N terminus was acetylated in order to keep it uncharged, in which the acetyl group on the N terminus is a protecting group. In another embodiment, the C terminus of SUPs was amidated in order to keep it uncharged, in which the amidated group on the C terminus is also a protecting group. The self-assembly ability of the SUP is attributed to the hydrophobicity of the aliphatic amino acids one one end and the hydrophilicity of polar amino acid one the other end, the N terminus and C terminus protecting groups are not essential for the self-assembly. Therefore, protecting groups other than acetyl and amidated groups function the same as long as they does not change the amphiphilic feature of the peptide.

In one embodiment, the N-terminal protecting group is a peptidomimetic molecule, including natural and synthetic amino acid derivatives, wherein the N-terminus of the peptidomimetic molecule may be modified with a functional group selected from the group consisting of carboxylic acid, amide, alcohol, aldehyde, amine, imine, nitrile, an urea analog, phosphate, carbonate, sulfate, nitrate, maleimide, vinyl sulfone, azide, alkyne, alkene, carbohydrate, imide, peroxide, ester, aryl, ketone, sulphite, nitrite, phosphonate, and silane. In one embodiment, the C-terminal protecting group is selected from
functional groups, such as polar or non-polar functional groups, such as (but not limited to)
   —COOH, —COOR, —COR, —CONBR or —CONRR′ with R and R′ being selected from the group consisting of H, unsubstituted or substituted alkyls, and unsubstituted or substituted aryls,
   —NH2, —OH, —SH, —CHO, maleimide, imidoester, carbodiimide ester, iso-cyanate;
small molecules,
   such as (but not limited to) sugars, alcohols, hydroxy acids, amino acids, vitamins, biotin;
linkers terminating in a polar functional group,
   such as (but not limited to) ethylenediamine, PEG, carbodiimide ester, imidoester;
linkers coupled to small molecules or vitamins,
   such as biotin, sugars, hydroxy acids.

The self-assembling tripeptides have demonstrated their ability to form hydrogels with nanofibrous architecture. However, their use as cell culture 3D scaffolds has been hindered by their high critical gelation concentration, long gelation time, and/or missing gel transparency.^{15, 33}

In one embodiment, the SUP in the present disclosure has at least 4 amino acids. Increasing the SUP length by just one amino acid compared to tripeptides facilitates hydrogel formation and avoids both synthesis complexity and monetary cost.

In one embodiment, the SUP comprises at least one aromatic amino acid or an aliphatic counterpart of an aromatic amino acid.

In one embodiment, the aromatic amino acid phenylalanine (F) is positioned between 1-2 aliphatic amino acids and one nonaromatic polar amino acid. In a preferred embodiment, F was the the third amino acid from the N terminus, because it is a well-known self-assembling inductor.

In one embodiment, F was replaced by its closest non-natural aliphatic relative amino acid cyclohexylalanine (Z). The aromatic interactions associated with an aromatic amino acid or a six-carbon ring in the aliphatic counterpart of an aromatic amino acid are important in the self-assembly, since it has been suggested that hydrophobicity and β-sheet propensity play a more significant role in the process than aromaticity.^{14,34}

In one embodiment, the aliphatic amino acids isoleucine (I) and valine (V) were placed in the first and second positions and the nonaromatic polar amino acid lysine (K) were placed in the fourth position. By having this arrangement, the amphiphilic structure of the peptide can be maintain.

In one embodiment, the N-terminus and C-terminus of both IVFK (SEQ ID NO. 1) and IVZK (SEQ ID NO. 2) SUPs were acetylated and amidated, respectively, to suppress the charges and facilitate the assembly.^{14,32-34}

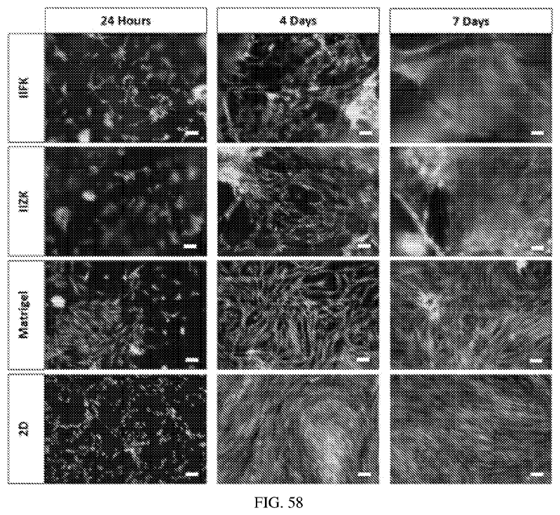

It was reported previously that an amphiphilic peptide could self-assemble if it passes a minimal hydrophobicity threshold. 55 The presence of an aromatic sidechain for 1π-stacking and an aromatic interaction can reduce the lag phase of aggregation kinetics, though it is not crucial for forming long-range fiber network which is needed for hydrogelation.^{34,56} FIG. 57 shows that the self-assembly rate of IIFK (SEQ ID NO. 33) is slower than the Cha-containing peptides (IIZK (SEQ ID NO. 34) and IZZK (SEQ ID NO. 35)), as it takes longer for IIFK (SEQ ID NO. 33) to assemble into gel at the concentrations of 1 mg/ml and 10 mg/ml, compared to IIZK (SEQ ID NO. 34) and IZZK (SEQ ID NO. 35). On the other hand, IZZK (SEQ ID NO. 35), which is more hydrophobic than IIZK (SEQ ID NO. 34) form a gel slower than IIZK (SEQ ID NO. 34) (FIG. 57), as it takes longer for IZZK (SEQ ID NO. 35) to form a gel at the concentrations of 1 mg/ml and 2 mg/ml, compared to IIZK (SEQ ID NO. 34).

In one embodiment, the self-assembly of SUP is impacted by the number and position of the aromatic amino acid or the aliphatic counterpart of an aromatic amino acid. Gelation of SUP with different number and position of the aromatic amino acid or the aliphatic counterpart of an aromatic amino acid are summarized below:

| Concentration | | IFFK | FFIK | | ZZIK | ZIIK | FIIK |
|---|---|---|---|---|---|---|---|
| 1 | mg/ml | >1 h | Does not | | — | <1 h | <15 min |
| | | Slightly | gel in 1 h | | | Very | Soft gel |
| | | soft gel | | | | soft | |
| | | | | | | gel | |
| 2 | mg/ml | <10 min | <10 | min | <10 min | <10 min | <5 min |
| | | | | | Soft gel | | |
| 3 | mg/ml | <5 min | <10 | min | <45 min | — | <10 min |
| | | | | | (<5 min | | |
| | | | | | Soft | | |
| | | | | | gel) | | |
| 4 | mg/ml | <5 min | — | | — | — | — |
| 5 | mg/ml | <5 min | <5 | min | <5 min | <10 min | <10 min |
| 10 | mg ml | <5 min | <5 | min | <5 min | <5 min | <5 min |
| 20 | mg/ml | <5 min | <5 | min | <5 min | <5 min | <5 min |

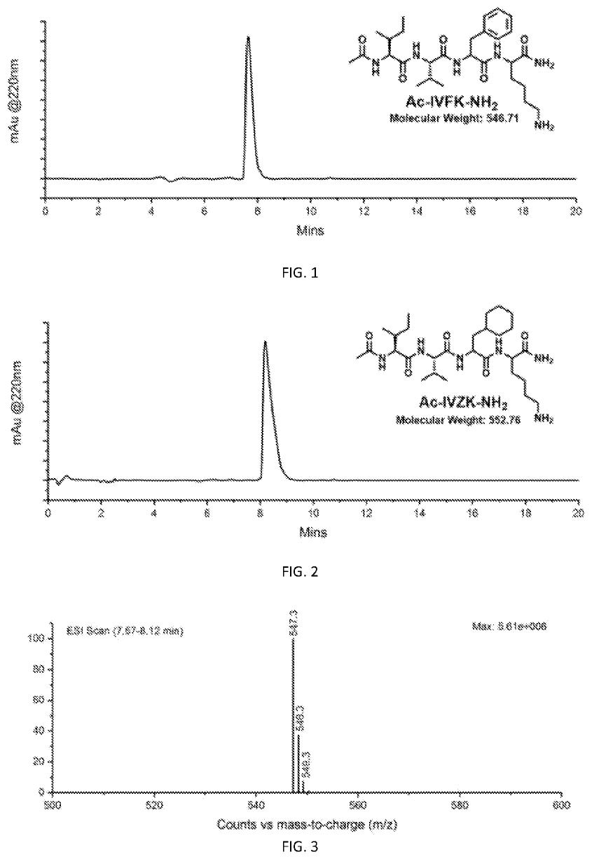

In one embodiment, the SUPs are IVFK (SEQ ID NO. 1) and IFZK. The structure of IVFK (SEQ ID NO. 1) and IFZK are shown in FIGS. 1 and 2, respectively. FIGS. 1 and 2 show the liquid chromatograms by the absorbance at 220 nm IVFK (SEQ ID NO. 1) and IFZK. Peptide purity from chromatograms was 96.64% and 97.50% for IVFK (SEQ ID NO. 1) and IVZK (SEQ ID NO. 2), respectively.

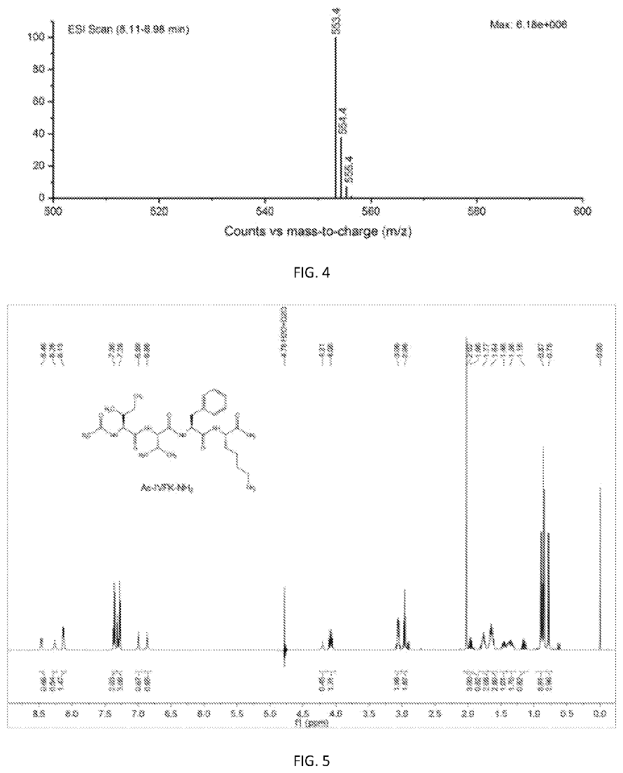

In one embodiment, IVFK (SEQ ID NO. 1) (m/z) calculated 546.7 and [M+H]⁺ found 547.3, while IVZK (SEQ ID NO. 2) (m/z) calculated 552.8 and [M+H]⁺ found 553.4, according to mass spectrum results as shown in FIGS. 3 and 4.

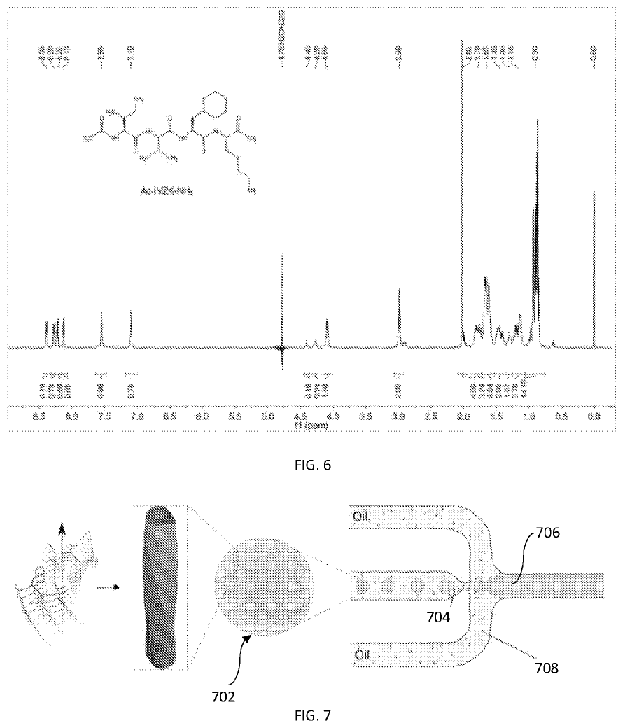

In one embodiment, ¹H NMR analysis of IVFK (SEQ ID NO. 1) is: ¹H NMR (600 MHz, Deuterium Oxide) δ 8.46 (d, J=7.1 Hz, 1H), 8.26 (d, J=7.7 Hz, 1H), 8.16-8.09 (m, 2H), 7.39-7.33 (m, 2H), 7.33-7.25 (m, 3H), 6.99 (s, 1H), 6.86 (s, 1H), 4.25-4.16 (m, 0H), 4.13-4.01 (m, 1H), 3.10-3.01 (m, 2H), 2.96 (t, J=8.1 Hz, 2H), 2.02 (s, 3H), 2.01-1.90 (m, 1H), 1.84-1.70 (m, 2H), 1.70-1.56 (m, 3H), 1.51-1.41 (m, 1H), 1.41-1.28 (m, 2H), 1.21-1.09 (m, 1H), 0.97-0.81 (m, 9H), 0.78 (d, J=7.0 Hz, 3H); and that of IVZK (SEQ ID NO. 2) is: ¹H NMR (600 MHz, Deuterium Oxide) δ 8.39 (d, J=7.2 Hz, 1H), 8.28 (d, J=7.5 Hz, 1H), 8.22 (d, J=8.6 Hz, 1H), 8.13 (d, J=7.5 Hz, 1H), 7.55 (s, 1H), 7.10 (s, 1H), 4.40 (s, OH), 4.34-4.20 (m, OH), 4.16-4.01 (m, 1H), 2.99 (t, J=8.3 Hz, 2H), 2.08-1.94 (m, 4H), 1.88-1.72 (m, 3H), 1.72-1.54 (m, 9H), 1.54-1.36 (m, 3H), 1.36-1.25 (m, 1H), 1.25-1.06 (m, 4H), 1.02-0.81 (m, 14H). The graph of ¹H NMR analysis is shown in FIGS. 5 and 6.

In one embodiment, elemental analysis of IVFK (SEQ ID NO. 1) and IVZK (SEQ ID NO. 2) is shown in the table below. Net peptide content (NPC)=(% Nmeasurement/% Ntheory)*100%.

| | | | | | Net peptide |
|---|---|---|---|---|---|
| | Elemental Analysis | % N | % C | % H | Content (%) |
| IVFK | Theoretical | 15.37 | 61.51 | 8.48 | 83.67 |
| | measurement | 12.86 | 53.62 | 6.97 | |
| IVZK | Theoretical | 15.2 | 60.84 | 9.48 | 85.59 |
| | measurement | 13.01 | 55.26 | 8.11 | |

In one embodiment, both peptides IVFK (SEQ ID NO. 1) and IVZK (SEQ ID NO. 2) harbor characteristic amphiphilic properties containing a hydrophobic tail and a hydrophilic headgroup.

In one embodiment, these peptides self-assemble noncovalently in aqueous solutions by molecular recognition via an antiparallel stacked fashion, forming fibers and bundles until they condense into a 3D nanofibrous network.^{14,15,20,21}

In one embodiment, the peptides are amphiphilic with three initial aliphatic amino acids capped by a polar lysine. This amphiphilic motif drives the peptide self-assembly through a βsheet antiparallel fashion, producing nanofibers, bundles, and a network that traps water as a 3D porous scaffold, forming a hydrogel with extracellular matrix-like topography. The nanofibers, bundles, and a network formed by IVFK (SEQ ID NO. 1) and IVZK (SEQ ID NO. 2) are illustrated in FIG. 7.

In one embodiment, these hydrogel features are kept in the microscale hydrogels fabricated by gelation in water-in-oil emulsion using a microfluidic flow-focusing chip. In the microfluidic chip, the SUP solution is focused and broken in microdroplets by the oil shear stress. Oil-dispersed sodium chloride diffuses and dissolves in the SUP microdroplets, triggering its gelation. This fabrication process is also illustrated in FIG. 7. In FIG. 7, the SUP solution 706 flows from right to left, while oil flows in the microfluidic flow-focusing chip through the top and bottom tubing and merges with the SUP solution 706. NaCl dispersed in oil 708 is shown as dots in the oil flow. After the oil merges with the SUP solution 706, some NaCl dissoves in SUP solution. The dissoved NaCl 704 triggers gelation, resulting in the formation of SUP microgel 702.

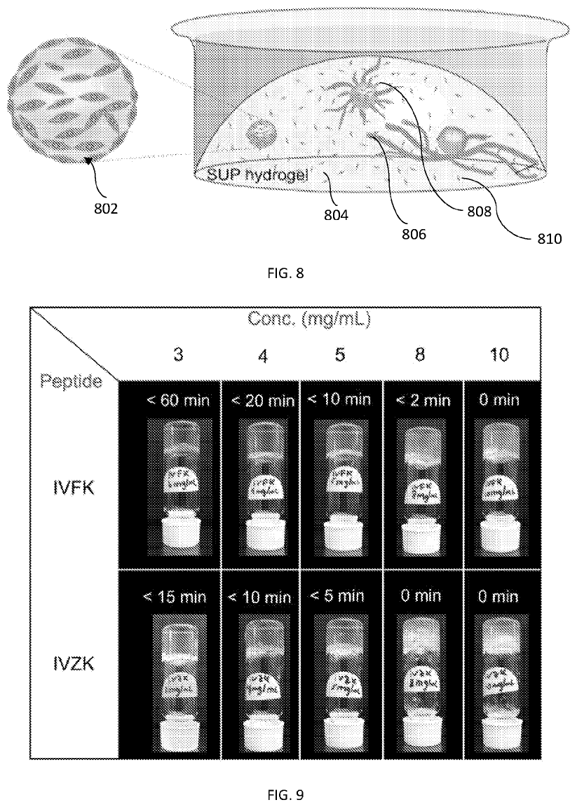

In one embodiment, SUP microgels are used as endothelial cell (EC) microcarriers where cells attach and proliferate. An individual EC-laden microgel 802 is illustrated in FIG. 8.

In one embodiment, the EC-laden microgels 802 are embedded within a 3D SUP hydrogel 804 loaded with fibroblasts 810 as the angiogenesis bead. ECs sprout from the microgel into the surrounding hydrogel, developing branched vessels with a lumen, as a mature vascular network. The sprouting EC 808 and EC with luman formation 806 are also illustrated in FIG. 8.

In one embodiment, both SUP IVFK (SEQ ID NO. 1) and IVZK (SEQ ID NO. 2) formed clear hydrogels at lower critical concentrations and shorter gelation times than their original tripeptides IVF and IVK.^{15,33} The hydrogels formed by IVFK (SEQ ID NO. 1) and IVZK (SEQ ID NO. 2) at different concentration and after different gelation time are shown in FIG. 9. As shown in FIG. 9, different concentrations of both peptides were tested at different time intervals to form a hydrogel in the presence of 1×PBS. 0 min indicates gelation right after mixing the PBS with the peptide solution.

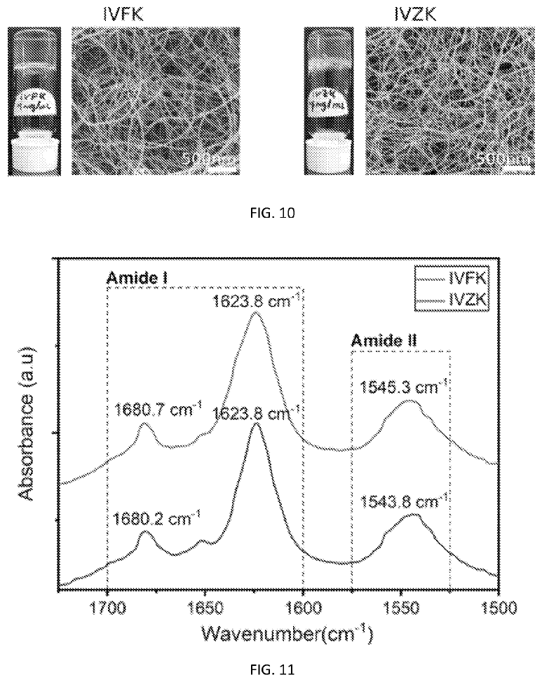

In one embodiment, IVFK (SEQ ID NO. 1) and IVZK (SEQ ID NO. 2) form hydrogel at 4 mg/mL after 10 min at room temperature in the presence of PBS. As shown in FIG. 10, both SUP hydrogels have an ECM-like topography as shown by scanning electron microscopy (SEM).

In one embodiment, IVFK (SEQ ID NO. 1) and IVZK (SEQ ID NO. 2) form β-sheet- and β-sheet-like structures. As shown in FIG. 11, the FTIR spectra of both SUPs show a similar profile with an evident peak at 1623 cm⁻¹, suggesting that both peptides have a β-sheet-like arrangement of the amide groups via intermolecular hydrogen bonding. In addition there is a second peak at 1680 cm⁻¹ that corresponds to either β-turn arrangement or an antiparallel β-sheet.^{30, 32}

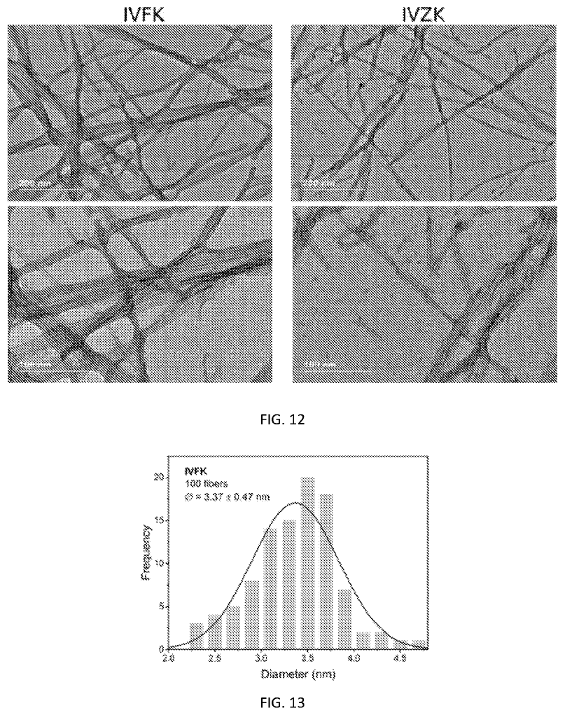

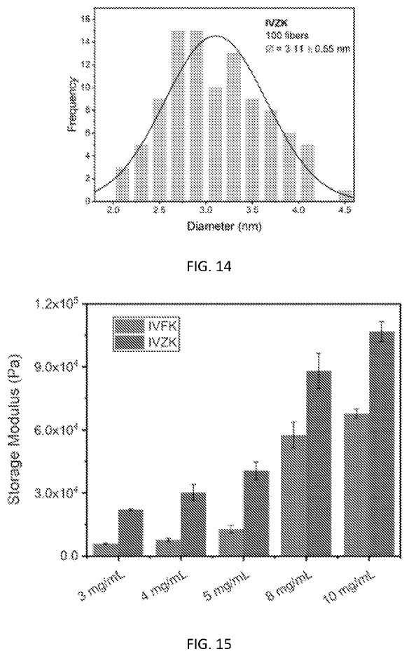

In one embodiment, the TEM imaging from both SUPs revealed a nanofibrous entangled network with long fibers forming bundles among them. The morphology of the nanofibrous network is shown in FIG. 12. In FIG. 12, the The red dash-line square in the top image encircles the area of the bottom image. The single-filament thickness is around 3 nm for both IVFK (SEQ ID NO. 1) and IVZK (SEQ ID NO. 2), as shown in FIGS. 13 and 14 respectively.

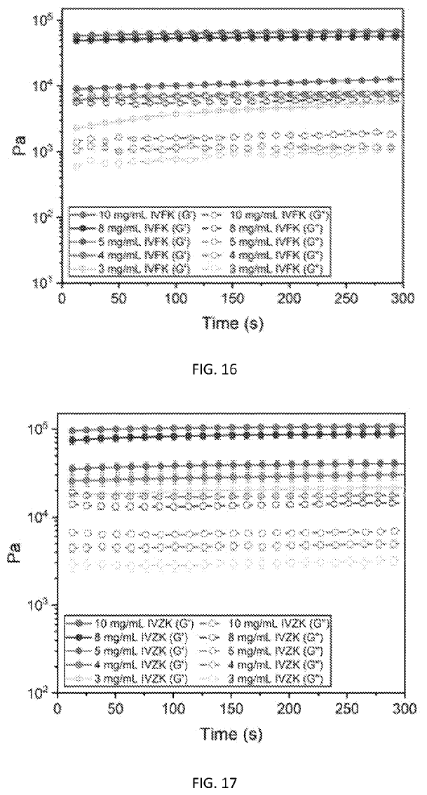

In one embodiment, the mechanical stiffness of the IVFK (SEQ ID NO. 1) and IVZK (SEQ ID NO. 2) hydrogels was directly proportional to the hydrogel concentration, in which IVZK (SEQ ID NO. 2) hydrogels showed higher stiffness than IVFK (SEQ ID NO. 1) hydrogels. The mechanical stiffness is measured as the storage modulus (G′) and loss modulus (G″). The storage moduli of IVFK (SEQ ID NO. 1) and IVZK (SEQ ID NO. 2) hydrogels at different concentrations are shown in FIG. 15, with the measurement taken after 5 min of time-sweep measurements at 0.1% strain and at 1 rads angular frequency (n=6). The values of G′ and G″ as a function of time or time sweep, strain or amplitude sweep, and angular frequency or frequency sweep are shown in FIGS. 16-21.

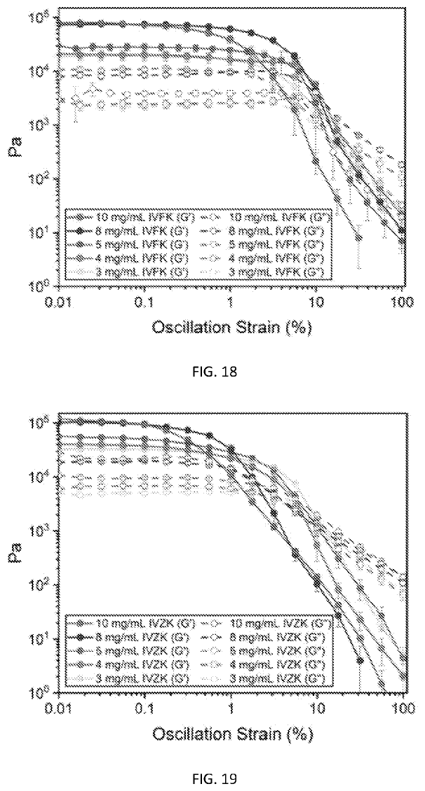

Despite having less stiffness reflected by lower G′ in FIG. 15 than the IVZK (SEQ ID NO. 2) hydrogel, the linear viscoelastic (LVE) region of the IVFK (SEQ ID NO. 1) hydrogel is wider than IVZK (SEQ ID NO. 2) as shown in FIGS. 18 and 19. The LVE region is the flat region of the curves in FIGS. 18 and 19, which reflects elastic of the hydrogels. This observation indicates that the IVFK (SEQ ID NO. 1) hydrogel is more elastic than the IVZK (SEQ ID NO. 2) hydrogel.³⁵

In one embodiment, the stiffness of the hydrogels can be tuned by adjusting the concentration. In a preferred embodiment, the stiffness of the hydrogels was tuned from 5 to 67 kPa for IVFK (SEQ ID NO. 1) and from 22 to 107 kPa for IVZK (SEQ ID NO. 2) by adjusting the peptide concentration from 3 to 10 mg/mL.

In one embodiment, IIFK (SEQ ID NO. 33), IIZK (SEQ ID NO. 34) and IZZK (SEQ ID NO. 35) shows similar valus of G′ and G″, as shown in the table below:

| | | Storage Modulus | Loss Modulus | tan δ |
|---|---|---|---|---|
| Peptide | Concentration | (G′, kPa) | (G′, kPa) | (G″/G′) |
| IIFK (SEQ | 1 mg/mL (1.78 mM) | 1.25 ± 0.86 | 0.13 ± 0.04 | 0.10 |
| ID NO. 33) | 2 mg/mL (3.56 mM) | 14.45 ± 1.37 | 1.31 ± 0.12 | 0.09 |
| | 5 mg/mL (8.90 mM) | 27.26 ± 1.30 | 2.39 ± 0.11 | 0.09 |
| | 8 mg/mL (14.2 mM) | 49.74 ± 5.02 | 5.00 ± 0.31 | 0.10 |
| | 10 mg/mL (17.8 mM) | 94.18 ± 7.12 | 9.04 ± 0.56 | 0.10 |
| | 13 mg/mL (23.1 mM) | 108.21 ± 5.02 | 8.90 ± 0.36 | 0.08 |
| IIZK (SEQ | 1 mg/mL (1.76 mM) | 6.52 ± 0.18 | 0.88 ± 0.04 | 0.13 |
| ID NO. 34) | 2 mg/mL (3.52 mM) | 16.82 ± 0.72 | 2.02 ± 0.09 | 0.12 |
| | 5 mg/mL (8.80 mM) | 64.74 ± 4.89 | 7.08 ± 0.59 | 0.11 |
| | 8 mg/mL (14.1 mM) | 89.40 ± 1.86 | 10.91 ± 0.44 | 0.12 |
| | 10 mg/mL (17.6 mM) | 134.04 ± 5.99 | 15.54 ± 0.68 | 0.12 |
| | 13 mg/mL (22.9 mM) | 271.34 ± 35.09 | 37.76 ± 2.38 | 0.14 |
| IZZK (SEQ | 1 mg/mL (1.65 mM) | 3.71 ± 0.19 | 0.36 ± 0.08 | 0.10 |
| ID NO. 35) | 2 mg/mL (3.30 mM) | 15.53 ± 0.74 | 1.44 ± 0.07 | 0.09 |
| | 5 mg/mL (8.25 mM) | 85.87 ± 6.50 | 11.60 ± 0.51 | 0.14 |
| | 8 mg/mL (13.2 mM) | 193.09 ± 10.13 | 29.70 ± 1.54 | 0.15 |
| | 10 mg/mL (16.5 mM) | 222.13 ± 8.16 | 39.71 ± 1.50 | 0.18 |
| | 13 mg/mL (21.5 mM) | 314.91 ± 10.10 | 54.56 ± 1.46 | 0.17 |

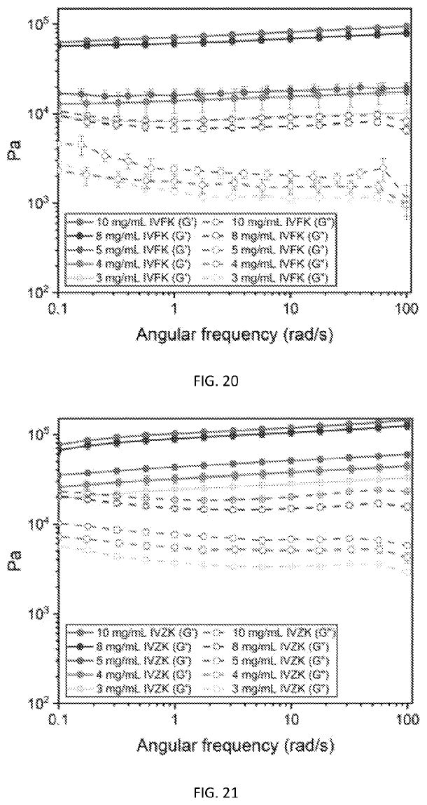

In one embodiment, a frequency-independent behavior was observed for both peptide hydrogels, as the storage modulus (G′) exhibited a plateau in the range 0.1-100 rads as shown in FIGS. 20 and 21. This characteristic is also commonly observed in hydrogels. 36

In one embodiment, IVFK (SEQ ID NO. 1) and IVZK (SEQ ID NO. 2) hydrogels were even stronger than other ultrashort amphiphilic peptides at the same molar concentration, as evidenced by the measurements described above.^{15, 18, 34}

### SUP Microgel Fabrication and Physical Characterization

In one embodiment, microgels made of tetrameric SUP, either IVFK (SEQ ID NO. 1) or IVZK (SEQ ID NO. 2), using a water-in-oil emulsion in a microfluidic droplet generator chip described above, were spherical.

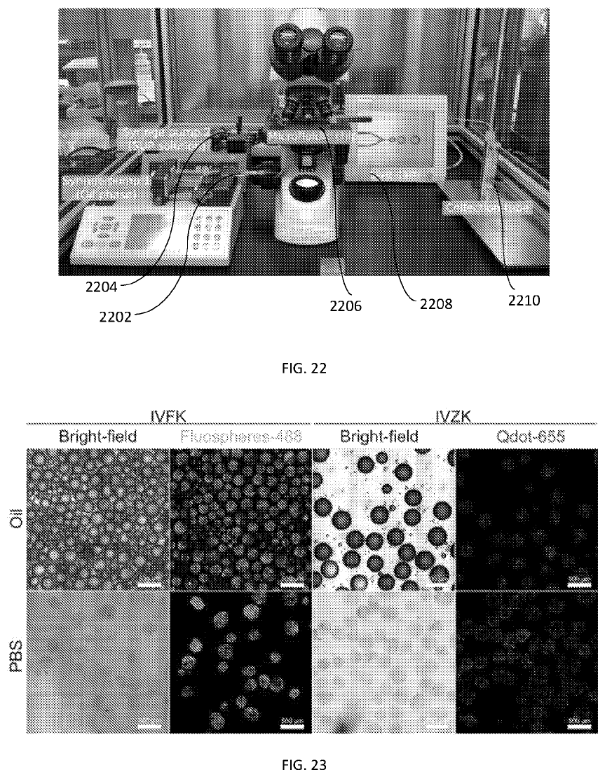

A prototype of the microfluidic setup is shown in FIG. 22, comprising a first syringe pump 2202 delivering oil phase, a second syringe pump 2204 delivering SUP solution, a microfluidic chip 2206 where the oil phase and SUP solution merge and the microgels form, a live cam 2208 that is used to monitor the formation of microgels, and a collection tube 2210. The microfluidic chip has a flow-focusing geometry to generate aqueous droplets by the oil-phase shear stress. Here, the aqueous phase is solely SUP dissolved in water (1.5 wt %), while the oil phase consisted of light mineral oil supplemented with both detergent Span 80 (2%) and dispersed sodium chloride fine powder (3 wt %). When the oil-dispersed sodium chloride gets into contact with the aqueous phase, it dissolves, forming ions that speed up the peptide self-assembly and trigger the gelation.^{15, 26} The spherical shape of these microgels are shown in FIG. 23.

In one embodiment, fluorescent nanoparticles were added in the SUP solution. In aqueous solution, the fluorescent nanoparticles would undergo Brownian motion. In one embodiment, fluorescent nanoparticle movement was not observed in the droplets once collected, indicating that the gelation already happened before the droplet came out from the pipes. In one embodiment, the nanoparticles did not move in a true SUP solution (1.5 wt %), highlighting the self-assembly of both tetrapeptides in water.

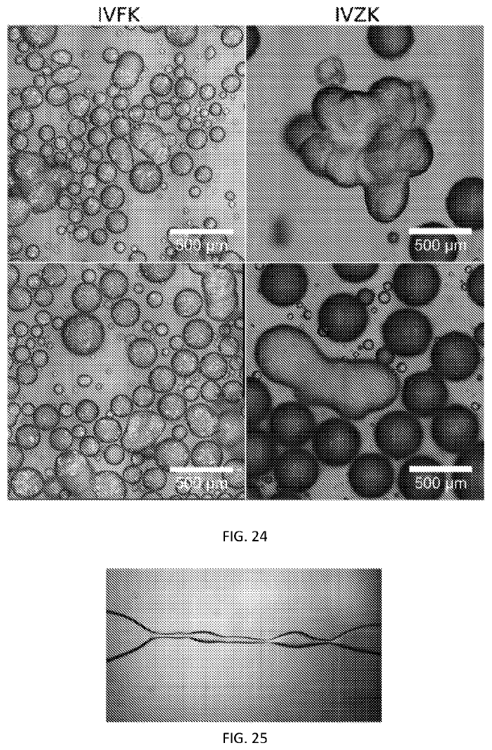

In one embodiment, sodium chloride in oil phase is required for the microgel integrity. The microgel fabrication was performed without the oil-dispersed sodium chloride and found some of these microgels merged with each other in the collection tube. FIG. 24 shows that both IVFK (SEQ ID NO. 1) and IVZK (SEQ ID NO. 2) microgels merged when sodium chloride is removed from the oil phase. The left and right columns show optical fluorescent image of 100 nm green FluoSpheres and 20 nm red fluorescent Qdots incorporated in IVFK (SEQ ID NO. 1) and IVZK (SEQ ID NO. 2) solution, respectively. This result indicates that the self-assembly in water is not enough to maintain the microgel integrity and the oil-dispersed sodium chloride is required to speed up the peptide self-assembly and trigger the gelation needed to keep the microgel integrity.

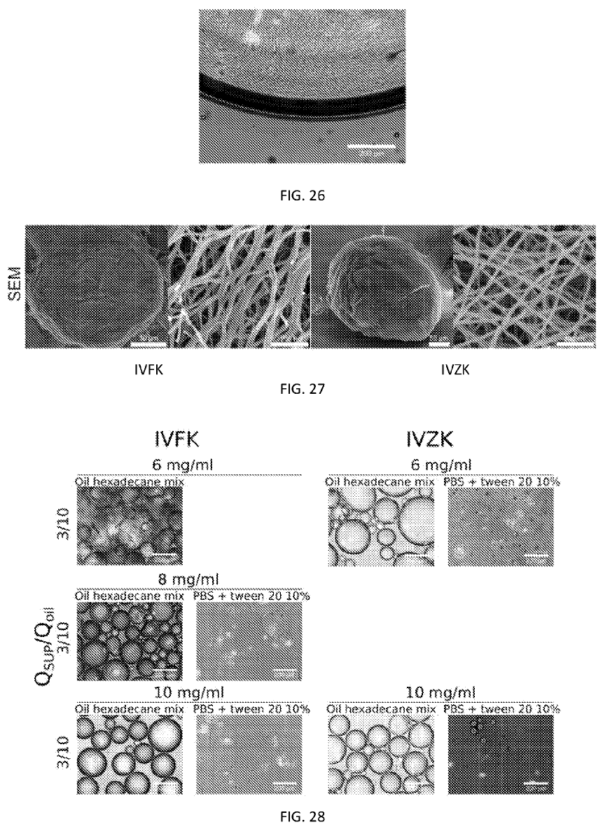

In another embodiment, surfactant in oil phase is also required for the microgel integrity. Removal of Span 80 from the oil mixture hinders droplet generation and produces a single bulky SUP mass in the collection tube, as shown in FIGS. 25 and 26. The use of surfactant Span 80 is meant to stabilize the droplets and improve droplet formation in a quick time frame, decreasing the aqueous dynamic surface tension and increasing the oil-phase shear.³⁷

In one embodiment, microgel fabrication using flow rates of Q SUP=10μL/min for SUP solution (1.5 wt %) and Q oil=1μL/min for light mineral oil supplemented with both detergent Span 80 (2%), and dispersed sodium chloride fine powder (3 wt %) produced spherical microgels with average diameters of 300 and 350μm for IVFK (SEQ ID NO. 1) and IVZK (SEQ ID NO. 2), respectively. The formation of microgels is shown in FIG. 23. In FIG. 23, bright-field images of the SUP microgels collected in oil and isolated in PBS are shown in the column of bright field; 100 nm green fluorescent FluoSpheres and 20 nm red fluorescent Qdots were incorporated in IVFK (SEQ ID NO. 1) and IVZK (SEQ ID NO. 2) solution, respectively. Scanning electron microscopy (SEM) of IVFK (SEQ ID NO. 1) and IVZK (SEQ ID NO. 2) microgels in FIG. 27 shows an ECM-like nanofibrous topography with a fiber thickness of around 20 nm. Both SUP microgel surfaces showed a nanofibrous network similar to the extracellular matrix with a fiber bundle thickness of around 20 nm.^{15, 16} Surprisingly, only IVZK (SEQ ID NO. 2) microgels at 1.5 wt % were stable enough to maintain their 3D spherical structure through SEM sample preparation using a critical point drier.

In one embodiment, increasing the SUP solution flow rate to Q SUP=2μL/min generated larger microgels that merged in the collection tube, while decreasing it to Q SUP=0.5μL/min stopped the microgel generation and even reversed the flow in the chip. In another embodiment, the microgel integrity was impacted by SUP concentration.

This inability to control the microgel diameter through flow rate changes might be attributable to the intrinsic properties of the peptide at high concentration (1.5 wt %), e.g., viscosity. Therefore, the SUP concentration was decreased until the microgel integrity was lost in the collection tube or during the isolation process. However, as SUP concentration decreased, the number of merged microgels increased in the collection tube due to slower gelation at a lower concentration.¹⁵ Regardless of the microgel merging grade after isolation in the aqueous phase, there were many intact microgels at minimum concentrations of 0.8 and wt % for IVFK (SEQ ID NO. 1) and IVZK (SEQ ID NO. 2), respectively. Effect of SUP concentration on microgel integrity us shown in FIG. 28. 100 nm FluoSpheres were used as a microgel tracer and ingetrity marker. Isolation process in oil/hexadecane mix and in PBS/tween are shown in FIG. 28. These results match with the higher critical gelation concentration in bulky hydrogels for IVFK (SEQ ID NO. 1) (0.4 wt %) compared to IVZK (SEQ ID NO. 2) (0.3 wt %).

In a preferred embodiment, the optimal parameters to fabricate SUP microgel were a peptide concentration of 1.5 wt %, mineral oil supplemented with both 2% Span 80 and dispersed sodium chloride fine powder of 3 mg/mL, and a flow rate ratio of Qoil/Q SUP=(μL/min).

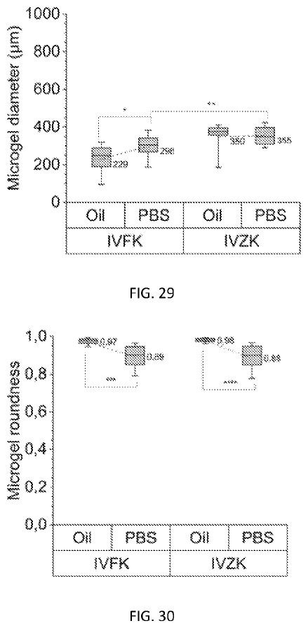

In one embodiment, the microgel extraction from the oil phase to the aqueous phase generated a swelling of 50μm in IVFK (SEQ ID NO. 1) microgels but not in IVZK (SEQ ID NO. 2) microgels. FIG. 29 compares the microgel diameter in oil phase and in aqueous phase (in PBS). In FIG. 29, box plots show percentile 25, 50, and 75 with whiskers at percentile 10 and 90. Dashed line connects the means depicted as squares and values. *indicates that IVFK (SEQ ID NO. 1) microgel diameter distribution differed between oil and PBS (MW, p<0.05). **indicates that Microgel diameter distribution in PBS differed between IVFK (SEQ ID NO. 1) and IVZK (SEQ ID NO. 2) (M-W p<0.05). The swelling difference suggests that IVFK (SEQ ID NO. 1) microgels are more hydrophilic and elastic than IVZK (SEQ ID NO. 2) microgels, in accordance with the higher stiffness of IVZK (SEQ ID NO. 2) bulky hydrogel over IVFK (SEQ ID NO. 1) shown in FIG. 15.

In one embodiment, the microgel extraction from the oil phase to the aqueous phase reduced the roundness of both IVFK (SEQ ID NO. 1) and IVZK (SEQ ID NO. 2) microgels from 0.98 to 0.88 on average, as shown in FIG. 30. In FIG. 30, box plots show percentile 25, and 75 with whiskers at percentile 10 and 90. Dashed line connects the means depicted as squares and values. ***indicates that IVFK (SEQ ID NO. 1) microgel roundness distribution differed between oil and PBS (M-W, p<0.05). ****indicates that IVZK (SEQ ID NO. 2) microgel roundness distribution differed between oil and PBS (M-W, p<0.05). Nonetheless, the SUP microgel roundness change could be negligible considering that roundness values go from 1.00 for a perfect circle to 0.00 for a very sharp and angular object.

In one embodiment, the SUP microgels can be used as a cell culture platform, as they are stabile under varying cell culture procedures such as autoclaving, UV irradiation, trypsinization, and rocking, at 37 and 22° C.

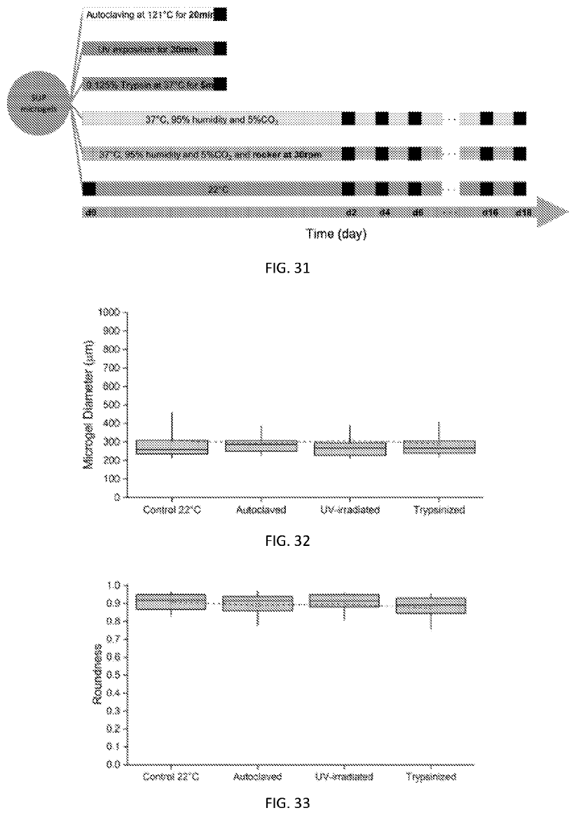

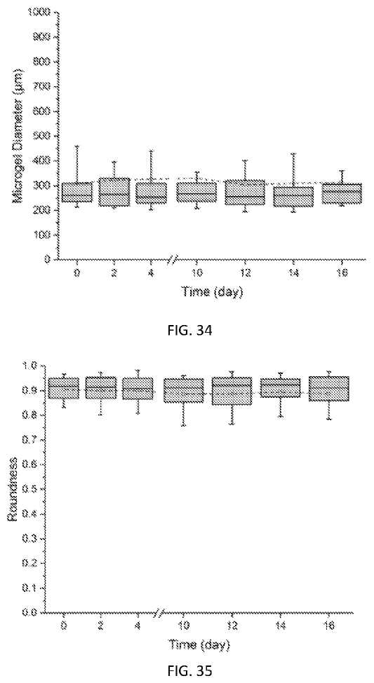

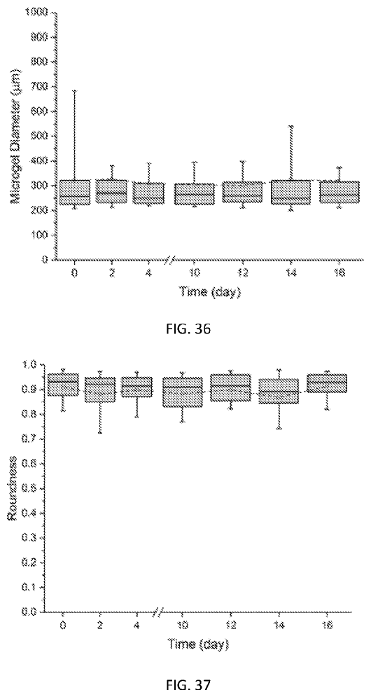

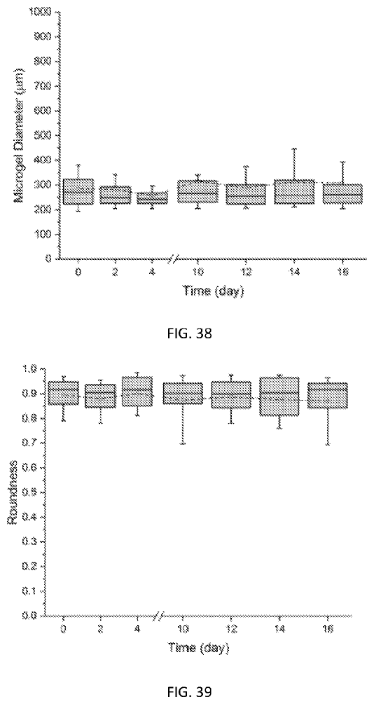

In one embodiment, both IVFK (SEQ ID NO. 1) and IVZK (SEQ ID NO. 2) microgels maintain their size and shape against challenges autoclaving, UV irradiation, trypsinization, and rocking, at 37 and 22° C. The challenges used to test the stability of IVFK (SEQ ID NO. 1) and IVZK (SEQ ID NO. 2) microgels include: (1) Autoclaving at 121° C. for min, (2) UV irradiation for 30 min, (3) trypsin at 37° C. for 5 min, (4) 37° C., 95% humidity and 5% CO₂, (5) 37° C., 95% humidity, 5% CO₂ and rocking at 30 rpm, and (6) 22° C. The duration and timeline of each challenges are illustrated in FIG. 31. In FIG. 31, the black square indicates the time-point of imaging. FIGS. 32-39 show the diameter and roundness of IVFK (SEQ ID NO. 1) microgel after different challenges, indicating the maintaining of stability. In FIGS. 32-39, the Box plots indicate percentile 25, 50, and 75 with whiskers at percentile 10 and The dashed line (---) connects the means depicted as squares. The distributions among the groups not differed significantly (Kruskal-Wallis, p>0.05). The statistical analysis of IVFK (SEQ ID NO. 1) microgel diameter and roundness stability against different treatments is summarized in the table below, in which DF refers to degrees of freedom and X2 refers to Chi-square.

| | Tested IVFK | | | | |
|---|---|---|---|---|---|
| | microgel | n per | | | |
| Compared treatments | variable | treatment | DF | X2 | p |
| 22 C., Autoclaving, | Diameter | 50 | 3 | 3.99 | >0.05 |
| UV and Trypsin | | | | | |
| 22 C., Autoclaving, | Roundness | 50 | 3 | 6.48 | >0.05 |
| UV and Trypsin | | | | | |
| 22 C. at day 0, 2, | Diameter | 50 | 6 | 1.69 | >0.05 |
| 4, 10, 12, 14, 16 | | | | | |
| 22 C. at day 0, 2, | Roundness | 50 | 6 | 1.65 | >0.05 |
| 4, 10, 12, 14, 16 | | | | | |
| 37 C. at day 0, 2, | Diameter | 50 | 6 | 1.28 | >0.05 |
| 4, 10, 12, 14, 16 | | | | | |
| 37 C. at day 0, 2, | Roundness | 50 | 6 | 7.52 | >0.05 |
| 4, 10, 12, 14, 16 | | | | | |
| 37 C. with mocking at day | Diameter | 50 | 6 | 5.81 | >0.05 |
| 0, 2, 4, 10, 12, 14, 16 | | | | | |
| 37 C. with rocking at day | Roundness | 50 | 6 | 3.62 | >0.05 |
| 0, 2, 4, 10, 12, 14, 16 | | | | | |

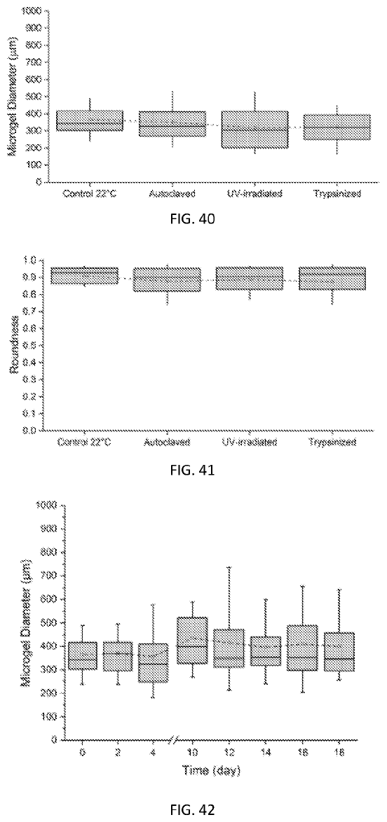

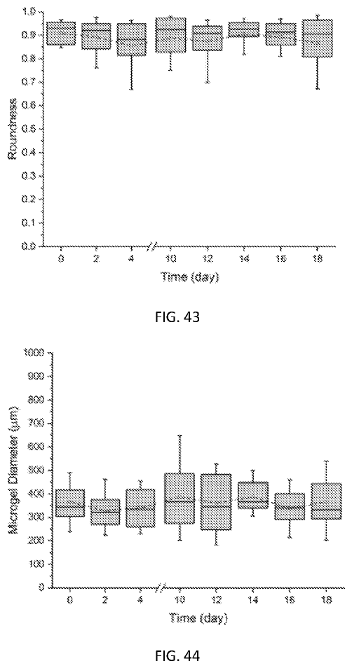

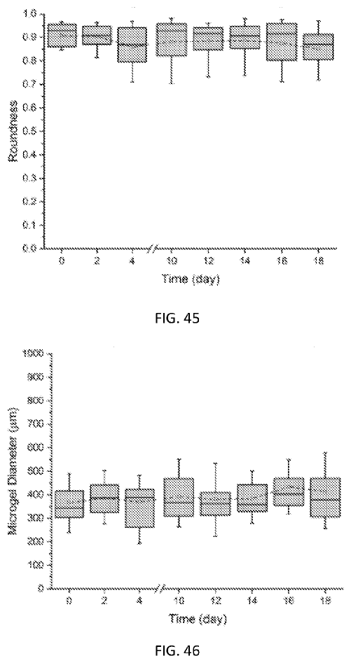

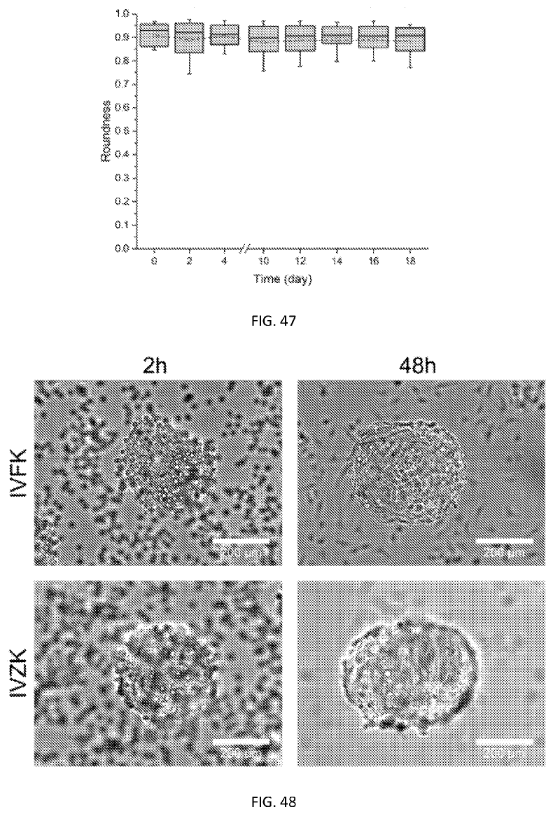

FIGS. 40-47 show the diameter and roundness of IVZK (SEQ ID NO. 2) microgel after different challenges, indicating the maintaining of stability. In FIGS. 40-47, the Box plots indicate percentile 25, 50, and 75 with whiskers at percentile 10 and 90. The dashed line (---) connects the means depicted as squares. The distributions among the groups not differed significantly (Kruskal-Wallis, p>0.05). The statistical analysis of IVZK (SEQ ID NO. 2) microgel diameter and roundness stability against different treatments is summarized in the table below, in which DF refers to degrees of freedom and X2 refers to Chi-square.

| | Tested IVFK | | | | |
|---|---|---|---|---|---|
| | microgel | n per | | | |
| Compared treatments | variable | treatment | DF | X2 | p |
| 22 C., Autoclaving, | diameter | 50 | 3 | 5.61 | >0.05 |
| UV and Trypsin | | | | | |
| 22 C., Autoclaving, | roundness | 50 | 3 | 1.82 | >0.05 |
| UV and Trypsin | | | | | |
| 22 C. at day 0, 2, 4, | diameter | 50 | 7 | 10.76 | >0.05 |
| 10, 12, 14, 16, 18 | | | | | |
| 22 C. at day 0, 2, 4, | roundness | 50 | 7 | 7.72 | >0.05 |
| 10, 12, 14, 16, 18 | | | | | |
| 37 C. at day 0, 2, 4, | diameter | 50 | 7 | 13.16 | >0.05 |
| 16, 12, 14, 16, 18 | | | | | |
| 37 C. at day 0, 2, 4, | roundness | 50 | 7 | 12.93 | >0.05 |
| 10, 12, 14, 16, 18 | | | | | |
| 37 C. with rocking at day | diameter | 50 | 7 | 10.89 | >0.05 |
| 0, 2, 4, 10, 12, 14, 16, 18 | | | | | |
| 37 C. with rocking at day | roundness | 50 | 7 | 4.28 | >0.05 |
| 0, 2, 4, 10, 12, 14, 36, 18 | | | | | |

The microgel stability against autoclaving (pressurized saturated steam at 121° C. for min) is unexpected, considering the noncovalent interactions that hold the assembled peptide network. This stability is ideal for developing products with a long shelf life that are ready to use in a cell culture.¹⁷

In one embodiment, the microgels have shown stable integrity for more than 6 months while being kept hydrated at 22° C. on a shelf. In addition, the microgel stability in the presence of trypsin and at 37° C. under agitation shows its potential for use as a microcarrier to grow cells in bioreactors.³⁸

In one embodiment, the factors that can be modified to tune the microgel properties, such as droplet size and microgel integrity, include: (1) sodium chloride and Span 80, (2) SUP concentration, (3) flow rate ratio, (4) several cell culture procedures, (5) chip geometry and (6) composition of the oil-dispersed salt, etc.

### SUP Microgels as Microcarriers

The use of microgels as microcarrier cell culture platforms offers several advantages over the typical adherent 2D culture systems. For instance, it increases the cell number in the culture because of its higher surface to volume ratio.³⁸ In addition, the microgel surface properties can be easily tuned to grow different cell types and control the cell behavior either by changing the microgel stiffness or by adding biochemical cues to mimic the native tissue.^{39, 40} Moreover, microgels can be used as an injectable delivery system for cell therapy due to their microsize and stability.⁴¹⁻⁴⁵

In one embodiment, the cells are grown on the microgel surface instead of encapsulating them because the cells multiply faster on the surface, and thus, higher cell numbers can be obtained. Although the cell encapsulation could resemble the 3D native environment in a better way, the encapsulation process could affect the cell viability considerably, since the cells must be included in the SUP solution and must tolerate the stringent microgel fabrication conditions.²⁶

In one embodiment, HeLa, HDFn, and HUVECs were grown on the surface of IVFK (SEQ ID NO. 1) and IVZK (SEQ ID NO. 2) microgels, adding only one cell type per microgel type at once.

In one embodiment, the SUP microgel is suitable as an in vitro culture platform.

First, adhesion of cells on the microgels is an important parameter in evaluating the suitability of microgels as a microcarrier. In one embodiment, HeLa cells adhered to the microgel as quick as 2 h and remained attached and stretched all around the microgel after 2 days of culture, as shown in FIG. 48. In FIG. 48, Hela cells were live stained with green tracker before seeding on the SUP microgels. In FIG. 48, the cells containing green florescent are living cells.

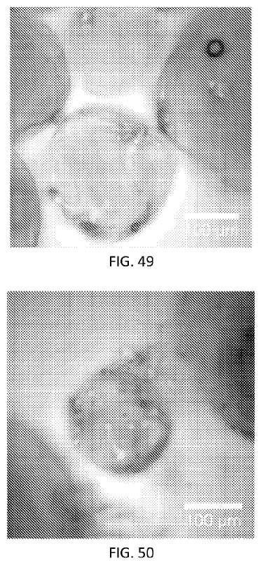

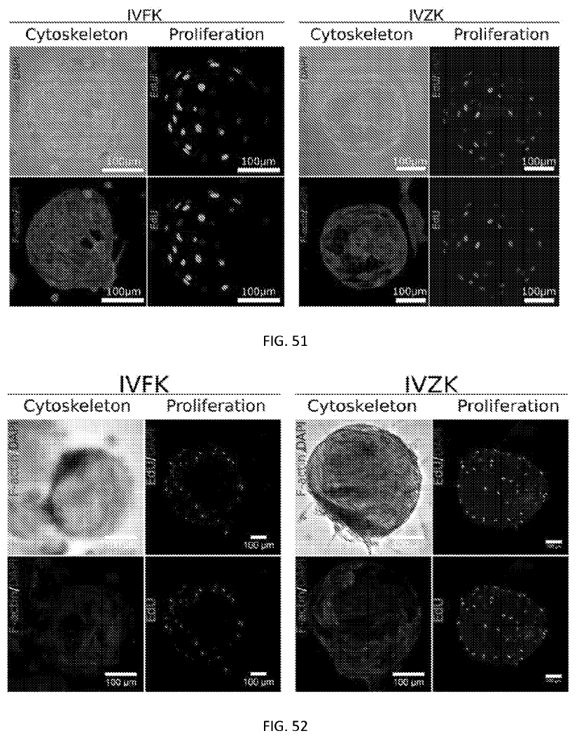

In another embodiment, two types of primary cells, fibroblasts (HDFn) and endothelial cells (HUVECs), were grown on the surface of IVFK (SEQ ID NO. 1) and IVZK (SEQ ID NO. 2) microgels at the same time, due to their well-known synergy to develop vascular networks in fibrin hydrogels.⁴⁶⁻⁴⁹ Both cell types showed cell adhesion and stretched morphology on both IVFK (SEQ ID NO. 1) and IVZK (SEQ ID NO. 2) microgels in the first 24 h of culture, as shown in FIGS. 49 and 50. In FIGS. 49 and 50, the images are maximum intensity projections of Z-stack imaging from representative cellladen SUP microgels.

In one embodiment, the cells proliferated all around the microgels after 8 days of culture, and most of the cells remained in a proliferative state at that time. FIG. 51 shows endothelial cells (HUVECs) cultured 8 days on a IVFK (SEQ ID NO. 1) or IVZK (SEQ ID NO. 2) microgel surface as a microcarrier in vitro culture platform. Cytoskeleton and proliferation staining of representative endothelial cell-laden SUP microgels. Images are maximum intensity projections of Z-stack imaging. Cytoskeleton is stained in red. Cytoskeleton top panel shows a merged image including bright field to visualize the SUP microgel. Proliferation top panel shows a merged image to visualize active nuclei (cyan) over total nuclei (blue). FIG. 52 shows fibroblasts (HDFn cells) cultured for 8 days on the SUP microgels surface as a microcarrier in vitro culture platform. The images are maximum intensity projections of Zstack imaging from representative HDFn cell-laden SUP microgels. Cytoskeleton is stained in red. The cytoskeleton top panel shows a bright-field included merged image to visualize the SUP microgel. The proliferation top panel shows a merged image to visualize active nuclei (cyan) over total nuclei (blue).

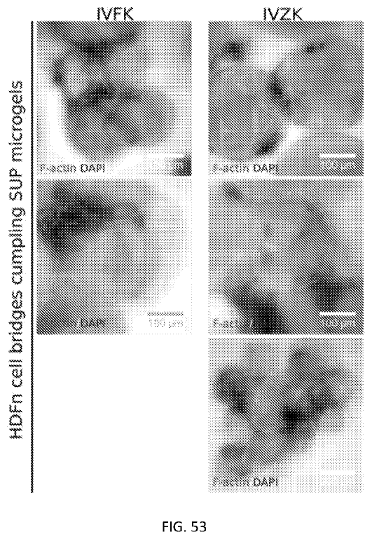

In one embodiment, HDFn formed cellular bridges connecting the microgels at longer culture periods. The cellular bridges formed by HDFn are similar to the cellular overgrowth seen in commercial microcarrier cultures.^{50, 51} As shown in FIG. 53, HDFn cells put the SUP microgels together by cell bridges when cultured for 9-20 days. The panel in FIG. 53 shows bright-field included merged images to visualize the SUP microgel and the fluorescent 3D cellular network. The images are maximum intensity projections of Z-stack imaging from representative clumped HDFn cell-laden SUP microgels. In FIG. 53, cytoskeleton is stained in red, while the nucleus is stained in blue.

In one embodiment, the grouped microgels interconnected by cells resembles the 3D growth and network formation seen in wound-healing microporous annealed particle scaffolds.⁴¹

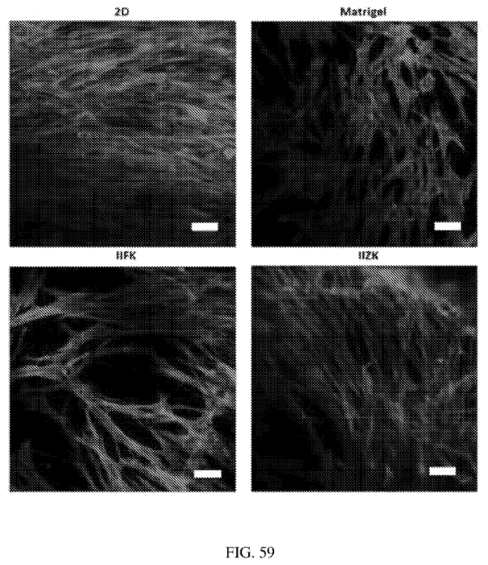

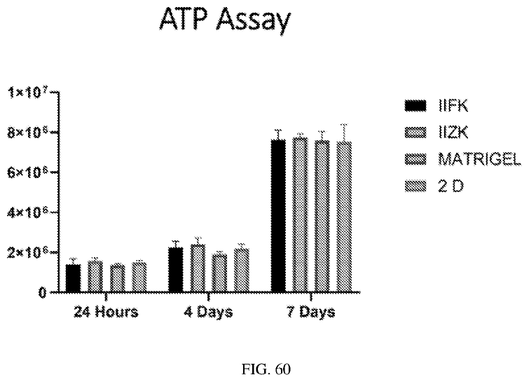

In one embodiment, human dermal fibroblasts (HDFn) are cultured within the 3D constructs formed by peptide hydrogels, and cell viability, metabolic activity, and morphology are analyzed. Upon 3D culturing, high cell viability and metabolic activity are confirmed. As shown in FIG. 58-59, the cells cultured in 3D hydrogel constructs are highly stretched and elongated, with well-defined actin fibers. FIG. 60 shows the proliferation of 3D cultured cells through quantitation of ATP production in metabolically active cells. There is an apparent change in cell morphology, as compared to 2D cultured cells, which was also reported by other studies.^{57, 58} The biocompatibility of a biomaterial, as indicated by cell viability, cell morphology and metabolic activity, is an essential factor for its potential use as a bioink and in regenerative medicine applications.

In one embodiment, SUP microgel can be used as a suitable microcarrier platform for different cell types.

### Endothelial Network Development by SUP Microgels Embedded in SUP Hydrogels

Blood vessel generation by vasculogenesis and angiogenesis is a process closely related to tissue development, remodeling, and repair. Therefore, unsuitable vascular development is characteristic of pathological conditions such as ischemia and cancer. Consequently, a better understanding will generate a positive impact on medicine, biology, and specifically in tissue engineering.^{28,29,46-49,52} However, both in vitro research models and therapeutic approaches rely on the use of natural materials such as collagen and fibrin. These materials have well-known limitations such as batch variability, immunogenicity, and infectious disease transmission risk, limiting its clinical applicability, especially for therapeutic implants.^{28, 29}

The bead assay is a 3D in vitro model for angiogenesis that encapsulates collagen-coated dextran microcarriers loaded with endothelial cells within a fibrin matrix with fibroblast cells on top to promote vessel formation.^{28, 29}

In one embodiment, the HUVEC-laden microgels described above can generate vascular networks within a SUP bulky hydrogel loaded with HDFn cells, in a similar way as the bead assay, although based solely on SUP and excluding the microcarrier collagen coating. The set up of vascular network generation using HUVEC-laden microgels in SUP bulky hydrogel loaded with HDFn cells is illustrated in FIG. 8.

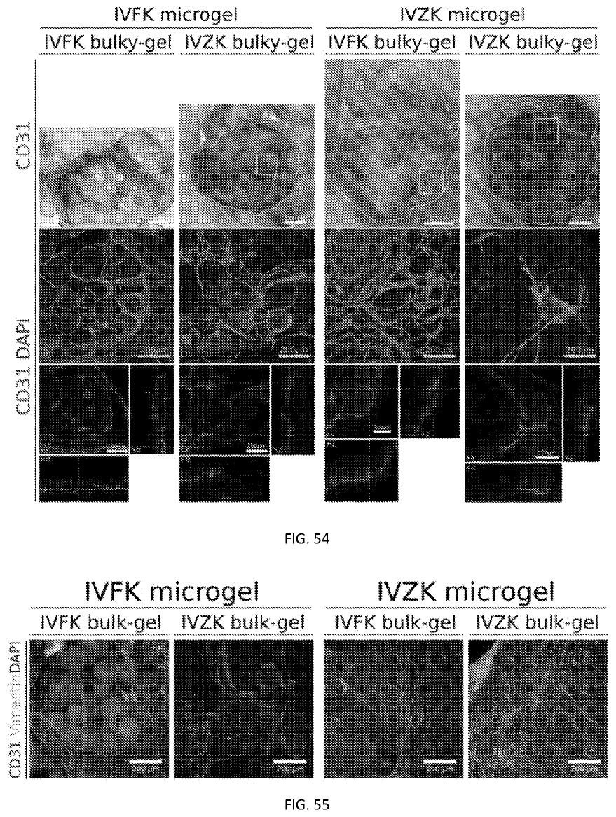

In one embodiment, the combinations between SUP bulky hydrogels (β) and SUP microgels (μ) include: β-IVFK (SEQ ID NO. 1)+μ-IVFK (SEQ ID NO. 1); β-IVFK (SEQ ID NO. 1)+μ-IVZK (SEQ ID NO. 2); β-IVZK (SEQ ID NO. 2)+μ-IVFK (SEQ ID NO. 1); β-IVZK (SEQ ID NO. 2)+μ-IVZK (SEQ ID NO. 2)). FIG. 54 shows the endothelial network formed using the above 4 combinations of SUP bulky hydrogels ((β) and SUP microgels (μ) after 20 days of coculture. In FIG. 54, endothelial cells are visualized with immune-staining of CD31 in red and the nucleus is stained with DAPI in blue. Top panel images are maximum intensity projections of whole mount tile scanning Z-stack imaging from SUP bulky hydrogels. White dotted line depicts the SUP hydrogel contour. Middle panel images are maximum intensity projections of Z-stack imaging from the squares in the top panel. White dashed line depicts the encapsulated SUP microgel contours. Bottom panel images are orthogonal sections of the confocal z-stacks from the middle panel images showing lumen formation. According to FIG. 54, both 1VFK (SEQ ID NO. 1) and IVZK (SEQ ID NO. 2) bulky hydrogels developed an evident endothelial network regardless of the SUP microgel used. However, the endothelial network developed in IVFK (SEQ ID NO. 1) bulky hydrogel looked more extended and complex than those observed in IVZK (SEQ ID NO. 2) bulky hydrogels.

In one embodiment, endothelial cells extended and migrated radially from the surface of the microgels into the peptide matrix, spanning across adjacent microgels and even distant ones.

In one embodiment, the endothelial network forms more complex structure in regions with multiple beads, where presumably the inosculation takes place connecting neighboring microgels.

In one embodiment, HDFn cells were distributed along the hydrogel in a layer-like fashion, colocalizing with both microgels and HUVECs cell in some spots. FIG. 55 shows vascular networks co-localize with fibroblast in 3D SUP matrices using a SUP microgel-based angiogenic in vitro assay. The 3D SUP hydrogels were stained with anti-CD31 Alexa Fluor 647 (red), anti-Vimentin Alexa Fluor 488 (green) and DAPI (blue) to detect endothelial cells, fibroblast cells and nuclei, respectively.

The lumen development is a vessel network maturation hallmark. Lumen development in the endothelial network may be determined using the orthogonal view of the confocal Zstack. 469 In one embodiment, lumen formation occurred in the developed endothelial networks in all bulky hydrogels, as shown in the bottom panel of FIG. 54. These multicellular branched hollow structures were more visible and also larger in IVFK (SEQ ID NO. 1) than IVZK (SEQ ID NO. 2) bulky hydrogels, suggesting once more the favorability of this SUP hydrogel to develop vascularized tissue constructs.

In one embodiment, the SUP microgels provided an endothelial cell monolayer that proliferates, sprouts radially, and branches into fibroblast-loaded SUP bulky hydrogels, producing a mature vascular network. This result resembles the process of angiogenesis, in which blood vessels are generated through sprouting and elongation of existing vasculature.⁵²

In one embodiment, the system modularity would allow it to adapt to resemble the vasculogenesis process, in which generated blood vessels de novo by mesodermal lineage cell coalescence into tubular structures.

In one embodiment, the microgels must be vascularized previously by a HUVECs/HDFn coculture at the microgel either on the surface or at the interior, or even both.^{40,47,48}

In one embodiment, the 3D in vitro assay based on SUP described above allows for testing how blood vessel formation is modulated by parameters such as cell type, cell ratio, cell location, matrix stiffness, and pore size.

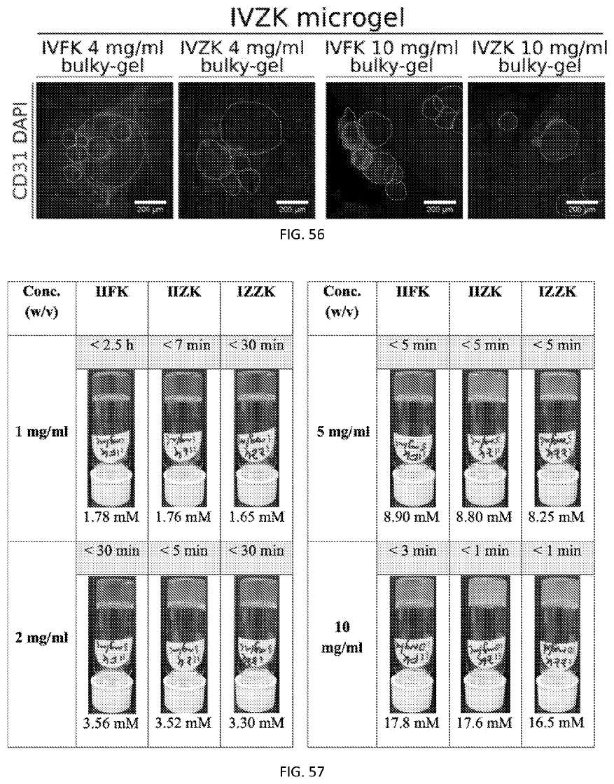

In one embodiment, stiffer SUP hydrogels (10 mg/mL) do not support endothelial network development as compared to softer SUP hydrogels (4 mg/mL), which show initial endothelial sprouting signs after 8 days in culture, invading the surrounding matrix. FIG. 56 shows the impact of bulk hydrogel stiffness on endothelial sprouting. In FIG. 56, endothelial cell-laden SUP microgels embedded in soft IVZK (SEQ ID NO. 2) bulk hydrogels (4 mg/ml) loaded with fibroblasts show initial signs of sprouting. The 3D SUP bulky hydrogels were stained with anti-CD31 Alexa Fluor 647 (red) and DAPI (blue) to detect endothelial cells and nuclei, respectively. The images are maximum intensity projections of Z-stack imaging.

In one embodiment, cell-laden microgels can be delivered by injection to support implantable cellular therapies and trigger vascularization and wound healing.⁴¹⁻⁴⁵

Several studies have proved this concept using natural materials, thus hindering the clinical translation. In one embodiment, the SUP-based system does not have such translational limitations, since it is based on synthetic but natural molecules that do not pose any infectious or immunoreactive riSk.^{17, 24, 25}

In one embodiment, SUP synthesis is highly reproducible and tunable, which makes SUP superb to natural origin materials.

In one embodiment, self-assembling tetrameric peptides IVFK (SEQ ID NO. 1) and IVZK (SEQ ID NO. 2) were used successfully to generate stable microgels. These peptides have been shown to self-assemble in nanofibrous networks, generating hydrogels with an ECM-like topography.

In one embodiment, micrometer-scale hydrogels or microgels can be fabricated from the self-assembled peptide networks using a water in oil emulsion, using a flow-focusing microfluidic droplet generator.

In one embodiment, the SUP microgels kept the ECM-like topography and maintained their size and shape during several cell culture procedures.

In one embodiment, microgels covered with cell lines, such as HeLa, HDFn, and HUVECs support continued cell culturing on the surface of the SUP microgels, demonstrating cell attachment, stretched morphology, and proliferation.

In one embodiment, the stability of the microgels allow their use as a favorable microcarrier platform for long-term cell culturing.

In one embodiment, HUVEC cells grown on the microgel surface and encapsulated in SUP bulky hydrogels loaded with HDFn demonstrated angiogenic potential of the SUP material. Thus, the SUP based system combining microgels and SUP bulk hydrogels can be used as a suitable cell-loaded microgel delivery system in vitro.

In one embodiment, the in vitro 3D tissue construct could be used as a model to study angiogenesis.

In one embodiment, the cell carrier microgels are suitable for cellular therapy use. The microgel's overall versatility and simplicity suggest interesting opportunities toward biomedical applications, for example, using them for pathological cardiovascular conditions and for the generation of vascularization in ischemic tissue.

Having described the many embodiments of the present disclosure in detail, it will be apparent that modifications and variations are possible without departing from the scope of the invention defined in the appended claims. Furthermore, it should be appreciated that all examples in the present disclosure, while illustrating many embodiments of the invention, are provided as non-limiting examples and are, therefore, not to be taken as limiting the various aspects so illustrated.

### EXAMPLES

### Example 1

Materials for Peptide Synthesis, Purification, LC-MS, and Elemental Analysis.

The designed peptides Ac-Ile-Val-Phe-Lys-NH2 (IVFK (SEQ ID NO. 1)) and Ac-Ile-Val-Cha-Lys-NH2 (IVZK (SEQ ID NO. 2)) were synthesized manually by solid phase peptide synthesis and purified by reverse phase-HPLC. The purity of the peptides IVFK (SEQ ID NO. 1) and IVZK (SEQ ID NO. 2) was >96% as calculated by HPLC data. 9-Fluorenymethoxycarbonyl (Fmoc) protected amino acids, MBHA rink amide resin, and (2-(1H-Benzotriazol-1-yl)-1,1,3,3-tetramethyluronium hexafluorophosphate (TBTU), hydroxy benzotriazole (HOBt) were purchased from GL Biochem, Shangai, China. N,N-diisopropylethylamine (DIPEA), piperidine, acetic anhydride, trifluoroacetic acid (TFA), Triisopropylsilane, N,N-dimethylformamide (DMF), dichloromethane (DCM), diethyl ether, and ethanol were purchased from Sigma-Aldrich®.

### Example 2

### Peptide Synthesis

The peptide sequences Ac-Ile-Val-Phe-Lys-NH2 (IVFK (SEQ ID NO. 1)) and Ac-(IVZK (SEQ ID NO. 2)) were synthesized manually on rink amide resin using the Fmoc-based solid-phase peptide synthesis (SPPS) method. The Fmoc group on resin was deprotected by 20% (v/v) piperidine/DMF prior to the first coupling of the amino acid. Amino acid coupling was performed by adding a mixture consisting of TBTU (3 equiv), HOBt (3 equiv), DIPEA (6 equiv), and Fmoc-protected amino acid (3 equiv) into the reaction vessel and agitated for 90 min. The Kaiser test was performed at the end of the coupling process to confirm completion of the coupling reaction. Acetylation on the N-terminus of peptide was performed by adding a mixture of 2:6:1 (v/v) acetic anhydride:DIPEA:DMF. Finally, the peptide was cleaved from the resin using a 95:2.5:2.5 mixture of TFA, water, and triisopropylsilane. The peptide in TFA solution was then dispersed in cold diethyl ether and kept standing overnight at 4° C. Then, the aggregated peptide was centrifuged and dried inside a vacuum desiccator. Peptide purification was then conducted in reversed-phase prep HPLC using a C-18 column. Both purified peptides were collected with more than 60% yield after being lyophilized.

### Example 3

### Liquid Chromatography-Mass Spectroscopy (LC-MS)

One milligram per milliliter of peptides in water was injected into an Agilent® 1260 Infinity LC equipped with an Agilent® 6130 Quadrupole MS and an Agilent® Zorbax® SB-C18 4.6×250 mm column; 0.1% (v/v) formic acid-water (A) and 0.1% (v/v) formic acid-acetonitrile (B) were chosen as the mobile phases. The flow of mobile phase was adjusted at 1.5 mL/min with a starting gradient of 98% A:2% B. In 18 min, the flow of B increased until 98% B and turned back to 2% again. LC chromatogram was obtained at a wavelength of 220 nm.

### Example 4

### ¹H Nuclear Magnetic Resonance

A 5 mg amount of each peptide was dissolved in 900 μL of 1 mM DSS in water. Then, 100 μL of D20 was added to each vial and mixed well. Spectra were recorded on a Bruker® Avance III 600 MHz equipped with a 5 mm Z-gradient SmartProbe BB(F)-H-D using a pulse program of zgesgp for water suppression.

### Example 5

### Elemental Analysis

Determination of the C, H, and N contents of both peptides was done using a Thermo Scientific® Flash 2000. A 2 mg amount of each peptide was weighted on a tin crucible, which was later used for wrapping the samples. Sulfanilamide was chosen as a calibration standard due to the nearest nitrogen percentage.

### Example 6

### Peptide Gelation Studies for Self-Assembly Characterization of Self-Assembling Ultra-short Peptide (SUP)

The preweighed (as per the required final concentration) peptide powder was dissolved in 0.9 mL of Milli-Q® water in a glass vial. The solution was vortexed for 5-10 s to obtain a clear and homogeneous solution. To this peptide solution, 0.1 mL of PBS buffer (10×, w/o Ca²⁺ and Mg²⁺) was added. To ensure a homogeneous distribution of buffer in the solution, the glass vial was further vortexed for 2-3 s. The glass vial was kept undisturbed on the bench, and hydrogel formation was observed using the vial inversion method, as shown in FIG. 9. The pH of the solutions of IVFK (SEQ ID NO. 1) and IVZK (SEQ ID NO. 2) was 5.57 and 5.48 at a 5 mg/mL concentration, respectively. After the addition of PBS, the pH changed to 7.7 and 7.6 for IVFK (SEQ ID NO. 1) and IVZK (SEQ ID NO. 2), respectively. The pH of the Milli-Q® water and phosphate buffer used was 6.7 and 7.4, respectively.

### Example 7

### Fourier-Transform Infrared Spectroscopy (FTIR)

Qualitative analysis of the assembled peptide structure was done to investigate the secondary structure of the peptide. First, the peptide solution was dropped on a glass coverslip and then dried overnight. The dried peptide then was analyzed using a Nicolet® iS10 FTIR equipped with a Smart iTR™ Diamond ATR accessory. The secondary structure was determined by performing peak deconvolution of the second derivative of the amide I band in the Origin software (OriginLab® Corp., Northampton, MA, USA).^{30,31} The second derivative of the FTIR spectrum was inverted by factoring by −1.

### Example 8

### Transmission Electron Microscopy (TEM)

TEM imaging was performed using a FEI Tecnai G2 Spirit Twin instrument with a 120 kV emission gun. One drop of diluted peptide hydrogel in water was placed on a carbon-coated copper grid that had been treated with glow discharge plasma prior to being used. The drop was kept on the grid for 10 min before being blotted using filter paper. The grid was stained with 2% uranyl acetate for 30 s to get better contrast. The grid was then rinsed in water to remove uranyl excess and dried for at least 1 day before imaging. The average diameter of the nanofibers was measure using hnageJ and Origin software from 100 fibers.

### Example 9

### Rheological Characterization of SUP Bulk Hydrogels

The mechanical stiffness of the peptide hydrogels was analyzed using a TA Ares-G2 Rheometer equipped with parallel-plate geometries of 8 mm diameter at room temperature. The sample gap between the upper and the lower geometries was set at 1.8 mm. The hydrogels were prepared from 135 μL of peptide solution that was mixed with 15 μL of 10×PBS in an 8 mm i.d. poly(methyl methacrylate) (PMMA) casting ring and left for 1 day before measurement. The rings were kept inside Petri dishes at room temperature with water surrounding them and tightly sealed to avoid dehydration. To control the accuracy of the measurements, six replicates for each peptide hydrogel were prepared. The stiffness was analyzed through three successive tests, which were time sweep, frequency sweep, and amplitude sweep. Time sweep was first performed for 5 min with an angular frequency and a strain of 1 rad/s and 0.1%, respectively. A frequency sweep was subsequently performed on the sample for a range of angular frequency of 0.1-100 rad/s with the same strain of 0.1%. The tests were completed with the amplitude sweep by applying a gradual increase of strain from to 100% at 1 rad/s angular frequency.

### Example 10

### Materials for General Cell Culture

Dulbecco's Modified Eagle Medium (DMEM), trypsin-ethylenediaminetetraacetic acid (EDTA) solution, fetal bovine serum (FBS, heat inactivated), penicillinstreptomycin, Dulbecco's phosphate-buffered saline (DPBS), neonatal human dermal fibroblasts (HDFn), cell culture incubator Forma II water jacket (5% CO₂, 95% humidity), and 50 mL polypropylene tubes with screw caps were bought from Thermo Fisher Scientific® Inc. Human umbilical vein endothelial cells (HUVEC), endothelial basal medium (EBM), and single quots kit were purchased from Lonza®. T75 cell culture flasks and 15 mL tubes with screw caps were bought from VWR®. The centrifuge 5810R was purchased from Eppendorf®. T25 cell culture flasks are from Greiner Bio-One®. Trypan blue solution 0.4% (w/v) in PBS was bought from Corning®. The hemocytometer was purchased from Hausser Scientific.

### Example 11

### Cell Culture

Frozen cryo-vials containing each cell line were thawed in a 37° C. water bath and diluted in complete cell culture medium (DMEM with 10% FBS, 100 units/mL of penicillin, 100μg/mL streptomycin for HDFn, HeLa, and EBM with singleQuots for HUVECs). Cells were seeded in a cell culture flask and incubated at 37° C., 95% humidity, and 5% CO₂. The medium was changed every other day until the cells were 80% confluent. At this confluency, the cells were split by removing medium, washing with DPBS, and adding trypsin/EDTA solution. After 5 min of incubation at 37° C., the cells detached completely and complete medium was added to inactivate trypsin. Cells were transferred to a tube and spun down at 300 g for 5 min. The supernatant was discarded, and the pellet was resuspended in 5 mL of complete medium. The cells were counted in a hemocytometer by the exclusion method using trypan blue and seeded at a concentration of 20,000 cells/cm² (100,000 cells/mL) for HDFn and HeLa or 2500 cells/cm² (12,500 cells/mL) for HUVECS. They were cultured as described until use or further splitting.

### Example 12

### Materials for Microgel Fabrication and Characterization

Hexadecane, Tween 20, Span 80, and sodium chloride were purchased from Sigma-Aldrich® Co. Light mineral oil, Dulbecco' s phosphatebuffered saline (DPBS), Biolite® 35 mm Petri dishes, green fluorescent FluoSpheres® 0.1 μm, and quantum dot (QD) 655 streptavidin were bought from ThermoFisher Scientific® Inc. A 40 mm soda lime glass Petri dish was bought from VWR®. An 18 gauge needle, 25 gauge syringe tip, and 1 (4.70 mm i.d.) and 3 mL (8.66 mm i.d.) luer-lock syringes came from Becton Dickinson Co. A 1 mL syringe BD luer1Lok tip (309628) and 3 mL syringe BD® luer1Lok tip (309657) were also used. Syringe pumps PHD and plus were purchased from Harvard Apparatus®. A syringe pump fusion 200 was bought from Chemyx. A flow-focused droplet generator (FFDG) 2.50 microfluidic chip pack, fluidic connect chip holder, tubing to pump connection kit, and Teflon tubing ( 1/16″ o.d., 250μm i.d.) were obtained from Micronit. A Milli-Q® water purification system was purchased from Millipore®. For droplet generation, on-site imaging using an Eclipse TS100 or E100 microscope equipped with a digital sight DS2Mv cam and a DSL2 controller from Nikon® were used. For microgel imaging, a LSM710 Zeiss® confocal microscope and BX-61 Olympus® polarized light microscope were used.

### Example 13

### Microgel Fabrication Using Flow-Focusing Microfluidic

Since the microgels are intended to be cell carriers, the fabrication materials were sterilized before use when possible and handled aseptically inside a biosafety cabinet (BSC) to reduce contamination risk. Milli-Q® water and hexadecane were sterilized by autoclaving at 121° C. for 20 min. Light mineral oil was sterilized by dry heating at 160° C. for 1 h. Peptide powder and sodium chloride were sterilized by UV irradiation (GE® germicidal lamp 30 W) for 30 min inside BSC. Once sterile, the peptide powder was dissolved to 27.1 mM (15 mg/mL) in sterile Milli-Q® water by vortexing for 5 min and incubated at room temperature (22° C.) for 1 h (preassembly). Span 80 was dissolved to 2% (v/v) into light mineral oil and hexadecane, independently. Sodium chloride was added to the light mineral oil+Span 80 mix and shaked to disperse the sodium chloride crystals. The peptide solution was loaded in a 1 mL syringe, while the light mineral oil with Span 80 2% (v/v) and sodium chloride 3 mg/mL were loaded in a 3 mL syringe. Both syringes were connected with the microfluidic system, and both solutions were brought to the droplet generator point by pressing the syringes manually. Then, both syringes were inserted into the pumps, and the flow rate was set at 10 μL/min for continuous phase (light mineral oil with Span 80 and sodium chloride) and 1 μL/min for dispersed phase (peptide solution). Droplet generation was monitored by on-site bright-field microscopy, and once it was stable and continuous, the droplets were collected in a tube containing light mineral oil with Span 80 2% (v/v). To confirm peptide droplet gelation, FluoSpheres® or QD were sometimes incorporated into the peptide solution during the preassembly incubation. Microgels were isolated from oil adding the same volume of hexadecane containing Span 80 (2% v/v) and centrifuged at 10 g for 5 min, and the oil layer was discarded. Microgels were washed with hexadecane plus Span 80 (2% v/v) two more times, discarding the hexadecane layer. A volume of DPBS containing Tween 20 (10% v/v) was gently added on the microgels through the collection tube walls and removed after 5 min. DPBS—Tween 20 washing was repeated 2 more times, decreasing the Tween 20 content to 1% v/v and 0.1% v/v sequentially. Finally, 250μL of DPBS was added on the microgels to keep them soaked, and they were kept on a shelf (22° C.) until further use. The microgel shape and size were checked by confocal laser scanning microscopy (CLSM).

### Example 14

Microgel Stability under Cell Culture Procedures

A 400 μL amount of microgels was diluted in 6 mL of DMEM, and aliquots of 1 mL were seeded in five 35 mm plastic Petri dishes and one 40 mm glass Petri dish. Each dish containing microgels was treated as follows: (1) Autoclaving at 121° C. for 20 min using the glass dish, (2) UV irradiation for 30 min, and (3) 0.125% trypsin-EDTA at 37° C. for 5 min. In addition, the remaining dishes containing microgels were treated for 16-18 days as follows: (4) 37° C., 95% humidity, and 5% CO₂, (5) 37° C., 95% humidity, and 5% CO₂ under rocker conditions at 30 rpm, and (6) 22° C. on a shelf. Microgel imaging was done before treatment and follow up every other day. FIG. 31 summarizes the experimental design.

### Example 15

### Microgel Imaging and Analysis

Microgel imaging was done at each condition and time point using a CLSM Zeiss® 710 or 880 by tile scanning with 15% overlapping and online stitching to capture as many microgels as possible from the dish. The Feret diameter and roundness of each microgel was calculated using ImageJ software by manually drawing up to 59 microgel contours per condition. All statistical data analysis and microgel descriptor plotting were conducted in Origin software. To assess the effect of the treatments on the microgel diameter and roundness, a Kruskal—Wallis test with Posthoc test pairwise Mann—Whitney U tests was applied.

### Example 16

### SEM Microgel Preparation and Imaging

The surface topography of the microgels was visualized using a FEI Magellan XHR Scanning Electron Microscope with an accelerating voltage of 3 kV. The SEM samples were prepared by dehydrating the peptide microgels in a gradually increasing ethanol concentration. The dehydrated microgels were then dried in a Leica EM CPD300. The dried peptides were sputter coated with 5 nm Jr before imaging.

### Example 17

### Additional Materials to the General Cell Culture for Cell Culture on Microgels

A Nunclon® sphere 24-well low binding plate, Nunc® 12 mm and 27 mm glass bottom dishes, CellTracker® Green CMFDA dye, a compact digital rocker, a Clickt-iT plus EdU imaging kit (Alexa® Fluor 488 picolyl azide), methanol-free 16% formaldehyde solution (w/v), magnesium chloride, sucrose, Triton® X-100, phosphate buffer saline (PBS), Tween 20, and sodium azide were purchased from ThermoFisher Scientific® Inc. A FAK100 Actin Cytoskeleton Focal Adhesion Staining Kit containing TRITC-conjugated phalloidin and 2-(4-amidinophenyl)-1H-indole-6-carboxamidine (DAPI) was bought from Millipore®.

### Example 18

### Green Cell Tracker Staining

In some examples, cells were stained with green cell tracker before seeding on the microgels to confirm cell adhesion by CLSM. To do so, complete cell culture medium was removed from the cell culture flask, and 10 μM CellTracker® Green CMFDA Dye in serum-free medium was added to stain the cells at 37° C. for 30 min. Subsequently, the staining solution was removed, and cells were counted and used straight away. CellTracker® Green staining is expected to last for at least 72 h.

### Example 19

### Cell Culture on Microgels

A 1.2×10⁶ amount of cells suspended in 4 mL of complete cell culture media (300,000 cells/mL) was mixed with 200 μL of microgels by pipetting. The mix was seeded at mL/well in a 24-well low binding plate and kept at 37° C., 95% humidity, and 5% CO₂ under rocker conditions at 30 rpm for 24 h. Then, cell-laden microgels were transferred into a tube by pipetting and spun down at 50 g for 5 min. The upper layer, containing cells not adherent to the microgels, was discarded. At this moment, cell-laden microgels were either resuspended with complete cell culture media to continue cell culture under rocker conditions or processed for staining.

### Example 20

### Cell Nuclei Staining and Cell Adhesion Imaging

The cell-laden microgels were fixed with 4% formaldehyde (v/v) in PBS and incubated for 30 min at 22° C. Later, formaldehyde was removed by centrifugation, and cell-laden microgels were washed twice with PBS in a similar way. Permeabilization buffer (6.3 mM MgCl₂, 233.7 mM sucrose, 0.5% (v/v) Triton® X-100 in PBS) was kept at −20° C. and thawed just before use. Cells on microgels were permeabilized adding cold permeabilization buffer for 5 min and washed twice with PBS as before. Next, cell nuclei were stained with 0.1 g/mL DAPI in PBS for 5 min at 22° C. and washed three times with PBS for 5 min. Finally, cell-laden microgels were transferred to 12 and 27 mm glass bottom dishes and imaged with a CLSM Zeiss® 710 or 880. Z-stack imaging was performed, and the maximum intensity projection was presented.

### Example 21

### Cytoskeleton Staining

Cell-laden microgels were fixed, permeabilized, and washed as before. Then, they were blocked with blocking buffer (5% (v/v) FBS, 0.1% (v/v) Tween 20, and 0.02% (w/v) sodium azide in PBS) for 30 min at 22° C. and washed twice with PBS. Actin filaments in the cytoskeleton were stained with 0.2μg/mL TRITC-conjugated Phalloidin in PBS for 1 h at 22° C. protected from light. Cell-laden microgels were washed three times with PBS, followed by cell nuclei staining as before. Cell-laden microgels were transferred to 12 and 27 mm glass bottom dishes and imaged with a CLSM Zeiss® 710 or 880. Z-stack imaging was performed, and the maximum intensity projection was presented.

### Example 22

### Cell Proliferation Staining

Proliferative cells were labeled by incubating cell-laden microgels with 10 mM EdU in complete medium for 24 h in rocking culture at 30 rpm. After incubation, medium was removed and cell-laden microgels were fixed with 3.7% formaldehyde (v/v) in PBS and incubated for 15 min at 22° C. Fixed cell-laden microgels were transferred into a tube by pipetting and spun down at 50 g for 5 min. Fixative was removed, and cell-laden microgels were washed twice with 3% Bovine Serum Albumin (BSA, w/v) in PBS. Then, cells were permeabilized with 0.5% (v/v) Triton® X-100 in PBS for 20 min at 22° C. and washed twice with 3% BSA (w/v) in PBS. EdU was detected following the manufacturer instructions by incubating cell-laden microgels with a Click-iT Plus reaction cocktail for 30 min at 22° C. protected from light. Subsequently, the reaction cocktail was removed, and cell-laden microgels were washed once with 3% BSA (w/v) in PBS. Afterward, the samples were processed for cytoskeleton staining as described above and always protected from light during incubation. Finally, cell-laden microgels were transferred to 12 and 27 min glass bottom dishes and imaged with a CLSM Zeiss® 710 or 880. Z-stack imaging was performed, and the maximum intensity projection was presented.

### Example 23

### Additional Materials for Bead Assay Based Solely on SUP as ECM Biomaterial

Normal goat serum was purchased from ThermoFisher Scientific® Inc. Anti-CD31 antibody [JC/70A](Alexa® Fluor 647) ab215912 and Anti-Vimentin antibody [EPR3776](Alexa® Fluor 488) ab185030 were purchase form Abeam®.

### Example 24

### 3D SUP Hydrogel Bead Assay Coculture

This assay was done by iterating the 4 combinations between SUP bulk-hydrogels ((3) and SUP microgels (p) (i.e., β-IVFK (SEQ ID NO. 1)+μ-IVFK (SEQ ID NO. 1); β-IVFK (SEQ ID NO. 1)+μ-IVZK (SEQ ID NO. 2); βIVZK (SEQ ID NO. 2)+μ-IVFK (SEQ ID NO. 1); β-IVZK (SEQ ID NO. 2)+μ-IVZK (SEQ ID NO. 2)). The next procedure is the same for all combinations using as an example β-IVFK (SEQ ID NO. 1)+μ-IVFK (SEQ ID NO. 1). HUVECs were cultured on μ-IVFK (SEQ ID NO. 1) for 24 h as described previously. By the bead assay seeding day, IVFK (SEQ ID NO. 1) powder for β-hydrogel was dissolved at 8 mg/mL in sterile water 1 h before seeding. Meanwhile, HDFn cells were harvested and resuspended in 2 xPBS at 1.2×10⁶ HDFn cells/mL, and simultaneously, HUVEC laden μ-IVFK (SEQ ID NO. 1) were spun down in a tube at 50 g for 5 min. The supernatant was discarded, and 200 μL of HUVEC-ladenμ-IVFK (SEQ ID NO. 1) was mixed with 200 μL of HDFn cells in 2×PBS. Immediately, 100 μL of IVFK (SEQ ID NO. 1) solution at 8 mg/mL was poured on the culture dish and mixed with 100 μL of HUVEC-laden μ-IVFK (SEQ ID NO. 1) with HDFn cells in 2×PBS. The 3D hydrogel bead assay coculture was incubated at 37° C. for 10 min to guarantee β-IVFK (SEQ ID NO. 1) gelation. The final concentration of β-IVFK (SEQ ID NO. 1) was 4 mg/mL. Afterward, 2 mL of EGM-2 (EBM supplemented with singleQuots) was added to each dish containing the 3D hydrogel bead assay coculture, and the medium was changed every other day until fixation for immunofluorescence on culture day 8 and day 21.

### Example 25

### Vascular Network Staining

The medium was removed from the 3D SUP hydrogel bead assay coculture samples and incubated with PBS at 37° C. for 15 min. Samples were fixed with 4% formaldehyde (v/v) in PBS for 30 min at 22° C. and washed with PBS for 15 min at 22° C. under rocking. Later, samples were detached from the bottom of the plate using a bent spatula and transferred to individual wells in a 24-well plate where they were permeabilized with 0.5 mL per well of (v/v) Triton® X-100 in PBS for 1 h at 22° C. under rocking. After, samples were blocked overnight at 4° C. in a wet chamber under rocking with 0.5 mL per well of 10% (v/v) goat serum in PBS containing 0.01% (v/v) Triton® X-100. From the next step and on, samples were protected from light all of the time. Samples were incubated for 36 h at 4° C. in a wet chamber under rocking with anti-CD31 Alexa® Fluor 647 and anti-Vimentin Alexa® Fluor 488, both diluted 1/100 in blocking buffer. Samples were washed with PBS for −8 h at 22° C. with shaking, replacing the PBS every hour. Then, cell nuclei were stained with 0.1 μg/mL DAPI in PBS for 5 min at 22° C. and washed three times with PBS for 5 min Finally, samples were transferred to 27 mm glass bottom dishes and imaged with a CLSM Zeiss® 710 or 880. Imaging of the entire sample was done by whole mount Z-stack tile scanning, and zoom-in images were Z-stack imaging with orthogonal views. Maximum intensity projections of the whole mount and zoom-in are presented.

It is intended that the invention not be limited to the particular embodiment disclosed herein contemplated for carrying out this invention, but that the invention will include all embodiments falling within the scope of the claims.

All documents, patents, journal articles and other materials cited in the present application are incorporated herein by reference.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

### REFERENCES

The following references are referred to above and are incorporated herein by reference:
1. WHO, Cardiovascular diseases (CVDs). World Health Organization 2017.
2. Briquez, P. S.; Clegg, L. E.; Martino, M. M.; Mac Gabhann, F.; Hubbell, J. A., Design principles for therapeutic angiogenic materials. Nature Reviews Materials 2016, 1 (1), 15006.
3. Khan, 0. F.; Sefton, M. V., Endothelialized biomaterials for tissue engineering applications in vivo. Trends in Biotechnology 2011, 29 (8), 379-387.
4. Murphy, S. V.; Atala, A., 3D bioprinting of tissues and organs. Nature Biotechnology 2014, 32 (8), 773.
5. Skylar-Scott, M. A.; Uzel, S. G. M.; Nam, L. L.; Ahrens, J. H.; Truby, R. L.; Damaraju, S.; Lewis, J. A. Biomanufacturing of Organ-Specific Tissues with High Cellular Density and Embedded Vascular Channels. Sci. Adv. 2019, 5 (9), No. eaaw2459.
6. Datta, P.; Ayan, B.; Ozbolat, I. T. Bioprinting for Vascular and Vascularized Tissue Biofabrication. Acta Biomater. 2017, 51, 1-20.
7. Patterson, J.; Martino, M. M.; Hubbell, J. A. Biomimetic Materials in Tissue Engineering. Mater. Today 2010, 13 (1), 14-22.
8. Vlahos, A. E.; Cober, N.; Sefton, M. V., Modular tissue engineering for the vascularization of subcutaneously transplanted pancreatic islets. Proceedings of the National Academy of Sciences 2017, 114 (35), 9337-9342.
9. Nichol, J. W.; Khademhosseini, A., Modular tissue engineering: engineering biological tissues from the bottom up. Soft Matter 2009, 5 (7), 1312-1319.
10. Khademhosseini, A.; Langer, R., Microengineered hydrogels for tissue engineering. Biomaterials 2007, 28 (34), 5087-5092.
11. Boekhoven, J.; Stupp, S. I., 25th anniversary article: supramolecular materials for regenerative medicine. Advanced Materials 2014, 26 (11), 1642-1659.
12. Annabi, N.; Tamayol, A.; Uquillas, J. A.; Akbari, M.; Bertassoni, L. E.; Cha, C.; Camci-Unal, G.; Dokmeci, M. R.; Peppas, N. A.; Khademhosseini, A., 25th anniversary article: Rational design and applications of hydrogels in regenerative medicine. Advanced Materials 2014, 26 (1), 85-124.
13. Thiele, J.; Ma, Y.; Bruekers, S. M.; Ma, S.; Huck, W. T., 25th anniversary article: designer hydrogels for cell cultures: a materials selection guide. Advanced Materials 2014, 26 (1), 125-148.
14. Hauser, C. A.; Deng, R.; Mishra, A.; Loo, Y.; Khoe, U.; Zhuang, F.; Cheong, D. W.; Accardo, A.; Sullivan, M. B.; Riekel, C., Natural tri-to hexapeptides self-assemble in water to amyloid (3-type fiber aggregates by unexpected α-helical intermediate structures. Proceedings of the National Academy of Sciences 2011, 108 (4), 1361-1366.
15. Loo, Y.; Lakshmanan, A.; Ni, M.; Toh, L. L.; Wang, S.; Hauser, C. A., Peptide bioink: self-assembling nanofibrous scaffolds for three-dimensional organotypic cultures. Nano Letters 2015, 15 (10), 6919-6925.
16. Arab, W. T.; Niyas, A. M.; Seferji, K.; Susapto, H. H.; Alexander, I.; Cima, I.; Hauser, C. A., Evaluation of Peptide Nanogels for Accelerated Wound Healing in Normal Micropigs. Front. Nanosci. Nanotech. 2018, 4 (4), 1-9.
17. Loo, Y.; Wong, Y.-C.; Cai, E. Z.; Ang, C.-H.; Raju, A.; Lakshmanan, A.; Koh, A. G.; Zhou, H. J.; Lim, T.-C.; Moochhala, S. M., Ultrashort peptide nanofibrous hydrogels for the acceleration of healing of burn wounds. Biomaterials 2014, 35 (17), 4805-4814.
18. Mishra, A.; Loo, Y.; Deng, R.; Chuah, Y. J.; Hee, H. T.; Ying, J. Y.; Hauser, C. A., Ultrasmall natural peptides self-assemble to strong temperature-resistant helical fibers in scaffolds suitable for tissue engineering. Nano Today 2011, 6 (3), 232-239.
19. Wu, E. C.; Zhang, S.; Hauser, C. A., Self-assembling peptides as cell-interactive scaffolds. Advanced Functional Materials 2012, 22 (3), 456-468.
20. Rauf, S.; Susapto, H. H.; Kahin, K.; Alshehri, S.; Abdelrahman, S.; Lam, J. H.; Asad, S.; Jadhav, S.; Sundaramurthi, D.; Gao, X.; Hauser, C. A. E. Self-Assembling Tetrameric Peptides Allow in Situ 3d Bioprinting under Physiological Conditions. J. Mater. Chem. B 2021, 9 (4), 1069-1081.
21. Susapto, H. H.; Alhattab, D.; Abdelrahman, S.; Khan, Z.; Alshehri, S.; Kahin, K.; Ge, R.; Moretti, M.; Emwas, A.-H.; Hauser, C. A. E. Ultrashort Peptide Bioinks Support Automated Printing of Large-Scale Constructs Assuring Long-Term Survival of Printed Tissue Constructs. Nano Lett. 2021, 21 (7), 2719-2729.
22. Jiang, W.; Li, M.; Chen, Z.; Leong, K. W., Cell-laden microfluidic microgels for tissue regeneration. Lab on a Chip 2016, 16 (23), 4482-4506.
23. Wan, J., Microfluidic-based synthesis of hydrogel particles for cell microencapsulation and cell-based drug delivery. Polymers 2012, 4 (2), 1084-1108.
24. Seow, W. Y.; Salgado, G.; Lane, E. B.; Hauser, C. A., Transparent crosslinked ultrashort peptide hydrogel dressing with high shape-fidelity accelerates healing of full-thickness excision wounds. Scientific Reports 2016, 6, 32670.
25. Wieczorek, M.; Abualrous, E. T.; Sticht, J.; Alvaro-Benito, M.; Stolzenberg, S.; Noe, F.; Freund, C., Major histocompatibility complex (MHC) class I and MHC class II proteins: conformational plasticity in antigen presentation. Frontiers in Immunology 2017, 8, 292.
26. Tsuda, Y.; Morimoto, Y.; Takeuchi, S., Monodisperse cell-encapsulating peptide microgel beads for 3D cell culture. Langmuir 2010, 26 (4), 2645-2649.
27. Tian, Y. F.; Devgun, J. M.; Collier, J. H., Fibrillized peptide microgels for cell encapsulation and 3D cell culture. Soft Matter 2011, 7 (13), 6005-6011.
28. Morin, K. T.; Tranquillo, R. T., In vitro models of angiogenesis and vasculogenesis in fibrin gel. Experimental Cell Research 2013, 319 (16), 2409-2417.
29. Nowak-Sliwinska, P.; Alitalo, K.; Allen, E.; Anisimov, A.; Aplin, A. C.; Auerbach, R.; Augustin, H. G.; Bates, D. O.; van Beijnum, J. R.; Bender, R. H. F., Consensus guidelines for the use and interpretation of angiogenesis assays. Angiogenesis 2018, 21 (3), 425-532.
30. Yang, H.; Yang, S.; Kong, J.; Dong, A.; Yu, S. Obtaining Information About Protein Secondary Structures in Aqueous Solution Using Fourier Transform Ir Spectroscopy. Nat. Protoc. 2015, 10 (3), 382-396.
31. Usoltsev, D.; Sitnikova, V.; Kajava, A.; Uspenskaya, M. Systematic Ftir Spectroscopy Study of the Secondary Structure Changes in Human Serum Albumin under Various Denaturation Conditions. Biomolecules 2019, 9 (8), 359.
32. Frederix, P. W.; Scott, G. G.; Abul-Haija, Y. M.; Kalafatovic, D.; Pappas, C. G.; Javid, N.; Hunt, N. T.; Ulijn, R. V.; Tuttle, T., Exploring the sequence space for (tri-) peptide self-assembly to design and discover new hydrogels. Nature Chemistry 2015, 7 (1), 30.
33. Lakshmanan, A.; Hauser, C. A., Ultrasmall peptides self-assemble into diverse nanostructures: morphological evaluation and potential implications. International Journal of Molecular Sciences 2011, 12 (9), 5736-5746.
34. Lakshmanan, A.; Cheong, D. W.; Accardo, A.; Di Fabrizio, E.; Riekel, C.; Hauser, C. A., Aliphatic peptides show similar self-assembly to amyloid core sequences, challenging the importance of aromatic interactions in amyloidosis. Proceedings of the National Academy of Sciences 2013, 110 (2), 519-524.
35. Tuncaboylu, D. C.; Argun, A.; Sahin, M.; Sari, M.; Okay, O. Structure Optimization of Self-Healing Hydrogels Formed Via Hydrophobic Interactions. Polymer 2012, 53 (24), 5513-5522.
36. Greenfield, M. A.; Hoffman, J. R.; Olvera de la Cruz, M.; Stupp, S. I. Tunable Mechanics of Peptide Nanofiber Gels. Langmuir 2010, 26 (5), 3641-3647.
37. Cha, C.; Oh, J.; Kim, K.; Qiu, Y.; Joh, M.; Shin, S. R.; Wang, X.; Camci-Unal, G.; Wan, K.-t.; Liao, R., Microfluidics-assisted fabrication of gelatin-silica core-shell microgels for injectable tissue constructs. Biomacromolecules 2014, 15 (1), 283-290.
38. Rodrigues, M. E.; Costa, A. R.; Henriques, M.; Azeredo, J.; Oliveira, R., Evaluation of solid and porous microcarriers for cell growth and production of recombinant proteins. In Animal Cell Biotechnology: Methods and Protocols; P8rtner, R., Ed.; Humana Press: Totowa, NJ, 2014; pp 137-147.
39. Allazetta, S.; Kolb, L.; Zerbib, S.; Bardy, J. a.; Lutolf, M. P., Cell-Instructive Microgels with Tailor-Made Physicochemical Properties. Small 2015, 11 (42), 5647-5656.
40. Allazetta, S.; Lutolf, M. P., Stem cell niche engineering through droplet microfluidics. Current Opinion in Biotechnology 2015, 35, 86-93.
41. Griffm, D R; Weaver, W. M.; Scumpia, P. O.; Di Carlo, D.; Segura, T., Accelerated wound healing by injectable microporous gel scaffolds assembled from annealed building blocks. Nature Materials 2015, 14 (7), 737-744.
42. Vegas, A. J.; Veiseh, 0.; Gildler, M.; Millman, J. R.; Pagliuca, F. W.; Bader, A. R.; Doloff, J. C.; Li, J.; Chen, M.; Olejnik, K., Long-term glycemic control using polymer-encapsulated human stem cell—derived beta cells in immune-competent mice. Nature Medicine 2016, 22 (3), 306.
43. Wang, L.; Rao, R. R.; Stegemann, J. P., Delivery of mesenchymal stem cells in chitosan/collagen microbeads for orthopedic tissue repair. Cells Tissues Organs 2013, 197 (5), 333-343.
44. Mahou, R.; Vlahos, A. E.; Shulman, A.; Sefton, M. V., Interpenetrating alginate-collagen polymer network microspheres for modular tissue engineering. ACS Biomaterials Science & Engineering 2018, 4 (11), 3704-3712.
45. Mahou, R.; Zhang, D. K.; Vlahos, A. E.; Sefton, M. V., Injectable and inherently vascularizing semi-interpenetrating polymer network for delivering cells to the subcutaneous space. Biomaterials 2017, 131, 27-35.
46. Newman, A. C.; Nakatsu, M. N.; Chou, W.; Gershon, P. D.; Hughes, C. C., The requirement for fibroblasts in angiogenesis: fibroblast-derived matrix proteins are essential for endothelial cell lumen formation. Molecular Biology of the Cell 2011, 22 (20), 3791-3800.
47. Peterson, A.; Caldwell, D.; Rioja, A.; Rao, R.; Putnam, A.; Stegemann, J., Vasculogenesis and angiogenesis in modular collagen—fibrin microtissues. Biomaterials Science 2014, 2 (10), 1497-1508.
48. Tiruvannamalai Annamalai, R.; Rioja, A. Y.; Putnam, A. J.; Stegemann, J. P., Vascular network formation by human microvascular endothelial cells in modular fibrin microtissues. ACS Biomaterials Science & Engineering 2016, 2 (11), 1914-1925.
49. Bezenah, J. R.; Kong, Y. P.; Putnam, A. J., Evaluating the potential of endothelial cells derived from human induced pluripotent stem cells to form microvascular networks in 3D cultures. Scientific Reports 2018, 8 (1), 1-14.
50. Borys, M. C.; Papoutsakis, E. T., Formation of bridges and large cellular clumps in CHO-cell microcarrier cultures: Effects of agitation dimethyl sulfoxide and calf serum. Cytotechnology 1992, 8 (3), 237-248.
51. Goh, T. K.-P.; Zhang, Z.-Y.; Chen, A. K.-L.; Reuveny, S.; Choolani, M.; Chan, J. K. Y.; Oh, S. K.-W., Microcarrier culture for efficient expansion and osteogenic differentiation of human fetal mesenchymal stem cells. BioResearch Open Access 2013, 2 (2), 84-97.
52. Potente, M.; Gerhardt, H.; Carmeliet, P., Basic and therapeutic aspects of angiogenesis. Cell 2011, 146 (6), 873-887.
53. Singh, Y.; Sharpe, P. C.; Hoang, H. N.; Lucke, A. J.; McDowall, A. W.; Bottomley, S. P.; Fairlie, D. P., Amyloid Formation from an A-Helix Peptide Bundle Is Seeded by 310-Helix Aggregates. Chem. Eur. J. 2011, 17 (1), 151-160.
54. Kayed, R.; Bernhagen, J.; Greenfield, N.; Sweimeh, K.; Brunner, H.; Voelter, W.; Kapurniotu, A., Conformational Transitions of Islet Amyloid Polypeptide (Tapp) in Amyloid Formation in Vitro. Edited by R. Huber. J. Mol. Biol. 1999, 287 (4), 781-796.
55. Betush, R. J.; Urban, J. M.; Nilsson, B. L. Peptide Science 2018, 110, (1), e23099.
56. Bowerman, C. J.; Ryan, D. M.; Nissan, D. A.; Nilsson, B. L. Molecular BioSystems 2009, 5, (9), 1058-1069.
57. Grinnell, F. Trends in cell biology 2003, 13, (5), 264-269.
58. Franco-Barraza, J.; Beacham, D. A.; Amatangelo, M. D.; Cukierman, E. Current protocols in cell biology 2016, 71, (1), 10.9. 1-10.9. 34.

1. A cell-laden microgel comprising:
at least one self-assembly ultrashort peptide (SUP) scaffold; and
at least one mammalian cells,
wherein the microgel has a spherical shape, and
wherein the diameter of the microgel is 100-900 μm.

2. The cell-laden microgel of claim 1, wherein the self-assembly ultrashort peptide (SUP) scaffold comprises at least one ultrashort peptide having a general formula selected from:
AₙBₘX, and XBₘAₙ
wherein the total number of amino acids of the ultrashort peptide does not exceed 7 amino acids;
wherein A is an aliphatic amino acids, selected from the group consisting of: isoleucine, norleucine, leucine, valine, alanine, glycine, homoallylglycine and homopropargylglycine or any combination thereof, with n being an integer being selected from 1-5;
wherein B is comprised of at least one aromatic amino acid selected from the group consisting of: tyrosine, tryptophan, phenylalanine, hydrophobic amino acid phenylalanine, or comprised of a peptidomimetic amino acid that is the aliphatic counterpart of the aromatic amino acid, such as cyclohexylalanine, which is the counterpart of amino acid phenylalanine with m being an integer being selected from 1-3;
wherein X is comprised of a polar amino acid, selected from the group consisting of: aspartic acid, glutamic acid, lysine, arginine, histidine, cysteine, serine, threonine, asparagine, and glutamine.

3. The cell-laden microgel of claim 1, wherein the self-assembly ultrashort peptide (SUP) scaffold comprises at least one peptide selected from the group consisting of:
(SEQ ID NO. 1)
IVFK,
(SEQ ID NO. 2)
IVZK,
(SEQ ID No. 3)
IVFD,
(SEQ ID No. 4)
IVZD,
(SEQ ID No. 5)
IVFE,
(SEQ ID No. 6)
IVZE,
(SEQ ID No. 7)
IVFS,
(SEQ ID No. 8)
IVZS,
(SEQ ID No. 9)
IVFR,
(SEQ ID No. 10)
IVZR,
(SEQ ID No. 11)
IVF(Dab),
(SEQ ID No. 12)
IVZ(Dab),
(SEQ ID No. 13)
IVF(Dap),
(SEQ ID No. 14)
IVZ(Dap),
(SEQ ID No. 15)
IVF(Orn),
(SEQ ID No. 16)
IVZ(Orn),
(SEQ ID No. 17)
KFVI,
(SEQ ID No. 18)
KZVI,
(SEQ ID No. 19)
DFVI,
(SEQ ID No. 20)
DZVI,
(SEQ ID No. 21)
EFVI,
(SEQ ID No. 22)
EZVI,
(SEQ ID No. 23)
SFVI,
(SEQ ID No. 24)
SZVI,
(SEQ ID No. 25)
RFVI,
(SEQ ID No. 26)
RZVI,
(SEQ ID No. 27)
(Dab)FVI,
(SEQ ID No. 28)
(Dab)ZVI,
(SEQ ID No. 29)
Dap)FVI,
(SEQ ID No. 30)
(Dap)ZVI,
(SEQ ID No. 31)
(Orn)FVI,
(SEQ ID No. 32)
(Orn)ZVI,
wherein I=isoleucine, L=leucine, V=valine, F=phenylalanine, K=lysine, D=aspartic acid, E=glutamic acid, S=serine, R=arginine, Z=cyclohexylalanine, (Dab)=2,4-diaminobutyric acid, (Dap)=2,3-diaminopropionic acid, and (Orn)=omithine.

4. The cell-laden microgel as claim 1, wherein self-assembly ultrashort peptide (SUP) scaffold has protecting group on at least one end,
wherein the protecting at the N terminus is N-terminal protecting group, and
wherein the protecting at the C terminus is C-terminal protecting group.

5. The cell-laden microgel of claim 4, wherein the N-terminal protecting group is a peptidomimetic molecule, including natural and synthetic amino acid derivatives, wherein the N-terminus of the peptidomimetic molecule may be modified with a functional group selected from the group consisting of carboxylic acid, amide, alcohol, aldehyde, amine, imine, nitrile, an urea analog, phosphate, carbonate, sulfate, nitrate, maleimide, vinyl sulfone, azide, alkyne, alkene, carbohydrate, imide, peroxide, ester, aryl, ketone, sulphite, nitrite, phosphonate, and silane.

6. The cell-laden microgel of claim 4, wherein the C-terminal protecting group is selected from
functional groups, such as polar or non-polar functional groups,
such as (but not limited to)
—COOH, —COOR, —COR, —CONBR or —CONRR′ with R and R′ being selected from the group consisting of H, unsubstituted or substituted alkyls, and unsubstituted or substituted aryls,
—NH2, —OH, —SH, —CHO, maleimide, imidoester, carbodiimide ester, iso-cyanate;
small molecules,
such as (but not limited to) sugars, alcohols, hydroxy acids, amino acids, vitamins, biotin;
linkers terminating in a polar functional group,
such as (but not limited to) ethylenediamine, PEG, carbodiimide ester, imidoester;
linkers coupled to small molecules or vitamins,
such as biotin, sugars, hydroxy acids.

7. The cell-laden microgel of claim 4, wherein the N-terminal protecting group is an acetylated group and the C-terminal protecting group is an amidated group.

8. The cell-laden microgel as in claim 1, wherein the mammalian cell is endothelial cells or fibroblast cells.

9. A microcarrier comprising cell-laden microgel as in claim 1.

10. A method of fabricating cell-laden microgel comprising:
feeding a microfluidic flow-focusing chip with at least one self-assembly ultra-short peptide (SUP) solution through a first inlet;
feeding a microfluidic flow-focusing chip with oil through a second inlet;
fabricating cell-free microgel using the microfluidic flow-focusing chip; and
loading the cell-free microgel with at least one mammalian cells,
wherein the oil comprising at least one selected from the group consisting of salt and surfactant,
wherein the microgel has a spherical shape, and
wherein the diameter of the microgel is 100-900 μm.

11. The method of claim 10, wherein the salt is NaCl.

12. The method of claim 10, wherein the concentration of salt is 3 mg/mL.

13. The method as in claim 10, wherein the surfactant is Span 80.

14. The method as in claim 10, wherein the concentration of surfactant is 2% (v/v).

15. The method as in claim 10, wherein the oil/SUP solution flow rate ratio is up to 10/1.

16. The method as in claim 10, wherein the SUP solution has a concentration of 1-9 mg/mL.

17. A cell culture system comprising:
at least one cell-laden microgels; and
at least one cell-loaded bulk hydrogels,
wherein the cell-laden microgels comprises a first self-assembly ultrashort peptide (SUP) scaffold and a first mammalian cell,
wherein the microgel has a spherical shape,
wherein the diameter of the microgel is 100-900 μm, and
wherein the cell-loaded bulk hydrogels comprises a second self-assembly ultra-short peptide (SUP) scaffold and a second mammalian cell.

18. The cell culture system of claim 17, where in the first mammalian cell and the second mammalian cell are at least one selected from the group consisting of endothelial cells or fibroblast cells.

19. The cell culture system of claim 17, wherein the first mammalian cell is endothelial cells.

20. The cell culture system as in claim 17, wherein the endothelial cells undergo sprouting and lumen formation that extends from the surface of the microgels into the bulk hydrogels.

21. The cell culture system as in claim 17, wherein the endothelial cells form endothelial network.

22. The cell culture system as in claim 17, wherein the first mammalian cell forms cell bridges that connect at least two nearby microgels.

23. A method of creating self-assembly ultrashort peptide (SUP) based cell culture system comprising:
feeding a microfluidic flow-focusing chip with a first self-assembly ultrashort peptide (SUP) solution through a first inlet;
feeding a microfluidic flow-focusing chip with oil through a second inlet;
fabricating cell-free microgel using the microfluidic flow-focusing chip;
loading the cell-free microgel with a first mammalian cells to create cell-laden microgels; and
dispersing the cell-laden microgels in a bulk hydrogel comprising a second self-assembly ultrashort peptide (SUP) scaffold and a second mammalian cell,
wherein the oil comprising at least one selected from the group consisting of salt and surfactant,
wherein the microgel has a spherical shape, and
wherein the diameter of the microgel is 100-900 μm.


SEQUENCE LISTING (35 sequences)

SEQ ID NO: 1 - protein, 4 aa, Artificial Sequence
  Synthetic
IVFK

SEQ ID NO: 2 - protein, 4 aa, Artificial Sequence
  Synthetic
  misc_feature (3)..(3): Xaa is cyclohexylalanine
IVXK

SEQ ID NO: 3 - protein, 4 aa, Artificial Sequence
  Synthetic
IVFD

SEQ ID NO: 4 - protein, 4 aa, Artificial Sequence
  Synthetic
  misc_feature (3)..(3): Xaa is cyclohexylalanine
IVXD

SEQ ID NO: 5 - protein, 4 aa, Artificial Sequence
  Synthetic
IVFE

SEQ ID NO: 6 - protein, 4 aa, Artificial Sequence
  Synthetic
  misc_feature (3)..(3): Xaa is cyclohexylalanine
IVXE

SEQ ID NO: 7 - protein, 4 aa, Artificial Sequence
  Synthetic
IVFS

SEQ ID NO: 8 - protein, 4 aa, Artificial Sequence
  Synthetic
  misc_feature (3)..(3): Xaa is cyclohexylalanine
IVXS

SEQ ID NO: 9 - protein, 4 aa, Artificial Sequence
  Synthetic
IVFR

SEQ ID NO: 10 - protein, 4 aa, Artificial Sequence
  Synthetic
  misc_feature (3)..(3): Xaa is cyclohexylalanine
IVXR

SEQ ID NO: 11 - protein, 4 aa, Artificial Sequence
  Synthetic
  misc_feature (4)..(4): Xaa is 24-diaminobutyric acid
IVFX

SEQ ID NO: 12 - protein, 4 aa, Artificial Sequence
  Synthetic
  misc_feature (3)..(3): Xaa is cyclohexylalanine
  misc_feature (4)..(4): Xaa is 24-diaminobutyric acid
IVXX

SEQ ID NO: 13 - protein, 4 aa, Artificial Sequence
  Synthetic
  misc_feature (4)..(4): Xaa is 23-diaminopropionic acid
IVFX

SEQ ID NO: 14 - protein, 4 aa, Artificial Sequence
  Synthetic
  misc_feature (3)..(3): Xaa is cyclohexylalanine
  misc_feature (4)..(4): Xaa is 23-diaminopropionic acid
IVXX

SEQ ID NO: 15 - protein, 4 aa, Artificial Sequence
  Synthetic
  misc_feature (4)..(4): Xaa is ornithine
IVFX

SEQ ID NO: 16 - protein, 4 aa, Artificial Sequence
  Synthetic
  misc_feature (3)..(3): Xaa is cyclohexylalanine
  misc_feature (4)..(4): Xaa is ornithine
IVXX

SEQ ID NO: 17 - protein, 4 aa, Artificial Sequence
  Synthetic
KFVI

SEQ ID NO: 18 - protein, 4 aa, Artificial Sequence
  Synthetic
  misc_feature (2)..(2): Xaa is cyclohexylalanine
KXVI

SEQ ID NO: 19 - protein, 4 aa, Artificial Sequence
  Synthetic
DFVI

SEQ ID NO: 20 - protein, 4 aa, Artificial Sequence
  Synthetic
  misc_feature (2)..(2): Xaa is cyclohexylalanine
DXVI

SEQ ID NO: 21 - protein, 4 aa, Artificial Sequence
  Synthetic
EFVI

SEQ ID NO: 22 - protein, 4 aa, Artificial Sequence
  Synthetic
  misc_feature (2)..(2): Xaa is cyclohexylalanine
EXVI

SEQ ID NO: 23 - protein, 4 aa, Artificial Sequence
  Synthetic
SFVI

SEQ ID NO: 24 - protein, 4 aa, Artificial Sequence
  Synthetic
  misc_feature (2)..(2): Xaa is cyclohexylalanine
SXVI

SEQ ID NO: 25 - protein, 4 aa, Artificial Sequence
  Synthetic
RFVI

SEQ ID NO: 26 - protein, 4 aa, Artificial Sequence
  Synthetic
  misc_feature (2)..(2): Xaa is cyclohexylalanine
RXVI

SEQ ID NO: 27 - protein, 4 aa, Artificial Sequence
  Synthetic
  misc_feature (1)..(1): Xaa is 24-diaminobutyric acid
XFVI

SEQ ID NO: 28 - protein, 4 aa, Artificial Sequence
  Synthetic
  misc_feature (1)..(1): Xaa is 24-diaminobutyric acid
  misc_feature (2)..(2): Xaa is cyclohexylalanine
XXVI

SEQ ID NO: 29 - protein, 4 aa, Artificial Sequence
  Synthetic
  misc_feature (1)..(1): Xaa is 23-diaminopropionic acid
XFVI

SEQ ID NO: 30 - protein, 4 aa, Artificial Sequence
  Synthetic
  misc_feature (1)..(1): Xaa is 23-diaminopropionic acid
  misc_feature (2)..(2): Xaa is cyclohexylalanine
XXVI

SEQ ID NO: 31 - protein, 4 aa, Artificial Sequence
  Synthetic
  misc_feature (1)..(1): Xaa is ornithine
XFVI

SEQ ID NO: 32 - protein, 4 aa, Artificial Sequence
  Synthetic
  misc_feature (1)..(1): Xaa is ornithine
  misc_feature (2)..(2): Xaa is cyclohexylalanine
XXVI

SEQ ID NO: 33 - protein, 4 aa, Artificial Sequence
  Synthetic
IIFK

SEQ ID NO: 34 - protein, 4 aa, Artificial Sequence
  Synthetic
  misc_feature (3)..(3): Xaa is cyclohexylalanine
IIXK

SEQ ID NO: 35 - protein, 4 aa, Artificial Sequence
  Synthetic
  misc_feature (2)..(3): Xaa is cyclohexylalanine
IXXK